(19) United States
(12) Patent Application Publication
(10) Pub. No.: US 2007/0031819 A1
(43) Pub. Date: Feb. 8, 2007

(54) Microfluidic systems for biological and molecular analysis and methods thereof

(75) Inventors: John Koschwanez, Seattle, WA (US); Mark Holl, Shoreline, WA (US); Robert Carlson, Seattle, WA (US); Michael McMurray, Berkeley, CA (US); Daniel E. Gottschling, Seattle, WA (US); Deirdre Ruth Meldrum, Mercer Island, WA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: University of Washington, Seattle, WA (US); Fred Hutchinson Cancer Research Center, Seattle, WA (US)

(21) Appl. No.: 11/412,819

(22) Filed: Apr. 26, 2006

Related U.S. Application Data
(60) Provisional application No. 60/674,851, filed on Apr. 26, 2005.

(51) Int. Cl. C12Q 1/00; G06K 9/00; C12M 1/34

(52) U.S. Cl. 435/4; 435/287.2; 382/128

(57) ABSTRACT

Disclosed herein are systems, components, devices and methods for automated yeast pedigree analysis. Systems, components, devices and methods for analyzing microorganisms, cells, particles and molecules are also disclosed.

### CROSS REFERENCE TO RELATED APPLICATIONS

This application claims the benefit of U.S. Provisional Application No. 60/674,851, filed Apr. 26, 2005, the entirety of which is incorporated by reference herein.

### STATEMENT OF GOVERNMENT RIGHTS

This invention was made with government support under R01 HG01497-06 and AG023779 awarded by the National Institutes of Health. The government has certain rights in the invention.

### FIELD OF THE INVENTION

The field of the invention pertains to microfluidic methods and devices for manipulating and analyzing microorganisms, cells, particles and molecules using magnetic forces. The field of the invention also pertains to methods of placing ferromagnetic structures on microfluidic substrates.

### BACKGROUND OF THE INVENTION

Cancer is caused by mutations in the genome that lead to unrestrained cell growth and colonization of other tissue by the resulting tumor. It is known that the incidence of cancer rises exponentially in the final decades of human life, but the exact mechanism by which age contributes to cancer is unknown. It is suspected that mutations could occur in genes responsible for maintaining genome stability, and these genes could accelerate the mutation rate [R. A. Depinho, “The age of cancer,” Nature, vol. 408, pp. 248-254, 2000]. The discovery of a mechanism that links deterioration in genomic stability and age could lead to the discovery of a mechanism that links higher incidence of cancer and age.

Loss of heterozygosity is a form of genetic instability. It occurs when the two alleles on a matching set of chromosomes are originally different (heterozygous), and then one of the alleles is either deleted or duplicated. Loss of heterozygosity is a frequent contributor to cancer in humans (for example, when the lost allele is the only copy of a tumor suppressor gene.) Researchers are currently searching for a link between loss of heterozygosity and age in a yeast cell.

Saccharomyces cerevisiae, or budding yeast, is a single-cell eukaryotic microorganism that commonly is used by biologists to study genetics. It is favored because it is easy to grow, the genome is well characterized, the cell cycle is short (less than 90 minutes), it can live as a haploid or diploid organism, and it is closely related to both plant and animal cells. A yeast cell will bud a daughter cell approximately every 70-90 minutes (see FIG. 1A). A mother's lifespan is defined as the number of times it buds, a total of 30-40 times on average [K. J. Bitterman, O. Medvedik, and D. A. Sinclair, “Longevity regulation in Saccharomyces cerevisiae: linking metabolism, genome stability, and heterochromatin,” Microbiol Mol Biol Rev, vol. 67, pp. 376-99, 2003].

A common tool of yeast geneticists is the insertion of a color marker gene into a yeast genome. The color will then show up as a visible phenotype under the correct conditions. For example, if the normal, dominant allele of the ADE2 gene is replaced by an ade2 mutant allele and grown in nutrient with adenine, the cell colony will turn red [H. Roman, “Studies of gene mutation in Saccharomyces,” Cold Spring Harb Symp Quant Biol, vol. 21, pp. 175-85, 1956]. If a diploid yeast cell is ADE2/ade2, it will form a white colony because the normal ADE2 allele is dominant. A loss of heterozygosity event in which the single ADE2 gene is deleted or replaced by ade2 will cause the cell to produce a red colony. MET15 is also used as a color marker. If MET15 is lost, the colony will turn brown on appropriate media [G. J. Cost and J. D. Boeke, “A useful colony colour phenotype associated with the yeast selectable/counter-selectable marker MET15,” Yeast, vol. 12, pp. 939-41, 1996].

In a known strain of yeast, ADE2 has been removed from its normal locations in the genome. A single copy of ADE2 is then placed at a locus near the telomere (the end of the chromosome) where loss of heterozygosity is likely to occur. Loci near the ends of chromosomes are more likely to experience loss of heterozygosity because typical mechanisms of loss of heterozygosity affect all loci between the initiating genetic lesion and the end of the chromosome. If the single copy of ADE2 is lost, the colony will turn red. A single copy of MET15 is similarly monitored. If the MET15 is lost, the colony will turn brown.

To analyze loss of heterozygosity as a function of age in a yeast cell, one can look for loss of heterozygosity in each daughter cell of a single mother cell. Capturing each of these daughters is done through an intensely manual process called yeast pedigree analysis.

In yeast pedigree analysis, a young mother cell is manually removed from a culture and then placed alone on an agar plate. The agar plate is placed on a moveable stage of a microscope, where the researcher watches the budding process. When the researcher sees a daughter cell bud off the mother, the researcher picks up the daughter cell using surface tension with a stationary glass fiber. The daughter cell is moved to a designated location on the agar plate corresponding to its bud generation number. The process is repeated every time the mother cell buds until the mother cell stops dividing. The agar plate is incubated to allow the daughter cells to grow into colonies, and genomic analysis is performed on each of the colonies. An agar plate with the grown daughter colonies, the final product of yeast pedigree analysis, is shown in FIG. 1B.

In a recent study [M. A. McMurray and D. E. Gottschling, “An age-induced switch to a hyper-recombinational state,” Science, vol. 301, pp. 1908-1911, 2003], McMurray and Gottschling performed yeast pedigree analysis using the ADE2/MET15 strain described above. If the daughter's colony was entirely red or brown, either the mother or the daughter lost heterozygosity upon division. If the colony was half red or half brown, one of the daughter's progeny lost heterozygosity after the first cell division; if the colony was quarter red or quarter brown, one of the daughter's progeny lost heterozygosity after the second cell division, and so on. The researchers could also tell whether heterozygosity was lost in the mother or the daughter. If every daughter subsequent to a loss of heterozygosity was red or brown, then mother lost heterozygosity. If subsequent daughters were white, then the daughter lost heterozygosity and the mother remained heterozygous.

McMurray and Gottschling found that loss of heterozygosity events (scored as daughter colony coloration of eighth sector or higher) were infrequent early in the mother's life. The first loss of heterozygosity event occurred at a median age of 23 buds. After the first event, the rate of loss of heterozygosity increased 40 to 200 times. This suggested a switch from a low genomic stability state to a high genomic stability state as the mother cell aged. They also found that heterozygosity was preferentially lost in the daughter cell. The cause of this genetic switch is still unknown; further research on loss of heterozygosity using yeast pedigree analysis is still needed. Unfortunately, this research is severely limited by labor requirements.

Yeast pedigree analysis is extremely labor intensive. During a recent pedigree analysis, one mother cell budded 100 daughter cells; the researcher was forced to spend almost 150 hours continuously in the lab. Due to these limitations, McMurray and Gottschling were able to analyze only two genes on the pedigrees of 40 mothers as part of their published study. The forty mothers were the same genotype and under the same environmental conditions. Expanding this research to analyze the remaining loci on every chromosome using multiple mutant strains under different environmental and nutritional conditions could not be performed in a researcher's lifetime using current methods.

Accordingly, automation of yeast pedigree analysis is currently needed. Such automation could help identify one or more mechanisms that cause the loss of heterozygosity. Identification of these mechanisms are expected to eventually lead to a greater understanding of human cancer. Automation will incorporate selective cell capture for the mother cell and free the daughter cell after budding. Cell capture methods include the magnetic capture of a biotinylated cells attached to streptavidin-coated paramagnetic beads. Lee et al., “Manipulation of biological cells using a microelectromagnet matrix,” Applied Physics Letters, vol. 85, pp. 1063-1065, 2004, has published the magnetic capture of a single cell using current-carrying conductors. Other groups have magnetically captured one or more paramagnetic beads [E. Mirowski, J. Moreland, S. E. Russek, and M. J. Donahue, “Integrated microfluidic isolation platform for magnetic particle manipulation in biological systems,” Applied Physics Letters, vol. 84, pp. 1786-1788, 2004; B. B. Yellen and G. Friedman, “Programmable assembly of colloidal particles using magnetic microwell templates,” Langmuir, vol. 20, pp. 2553-2559, 2004; T. Deng, G. M. Whitesides, M. Radhakrishnan, G. Zabow, and M. Prentiss, “Manipulation of magnetic microbeads in suspension using micromagnetic systems fabricated with soft lithography,” Applied Physics Letters, vol. 78, pp. 1775-1777, 2001].

### SUMMARY OF THE INVENTION

The present invention provides systems, components, devices, and methods that are useful in automating yeast pedigree analysis. For example, certain aspects of the present invention provide systems that include a microfluidic device comprising one or more magnetic capture sites optically coupled to an imaging system, wherein the magnetic capture sites are each capable of magnetically capturing a magnetically-labeled mother cell; each of the magnetic capture sites fluidically coupled to one or more collection receptacles; wherein the imaging system is capable of detecting the generation of a daughter cell by the magnetically-labeled mother cell and actuating the microfluidic device to fluidically transport the daughter cell to the one or more collection receptacles.

The present invention also provides systems, components, devices, and methods that have uses in addition to yeast pedigree analysis. For example, the systems, components, devices, and methods as provided herein are also useful in analyzing a variety of microorganisms, cells, particles and molecules. Accordingly, certain aspects of the present invention provide microfluidic devices that comprise a magnetic capture site capable of magnetically capturing a magnetically-labeled microorganism, cell, particle, molecule, or any combination thereof, the magnetic capture site comprising: one or more microchannels disposed on the microfluidic device; and one or more ferromagnetic structures disposed on or within the microfluidic device, the ferromagnetic structures comprising a tapered end and a body, wherein the tapered end of at least one of the ferromagnetic structures is proximately located to one or more lumens of one or more of the microchannels, the ferromagnetic structures capable of being magnetized using a magnetic field source proximately located external to the body of the ferromagnetic structure.

Various methods and components are also provided in preparing microfluidic devices that have a number of uses including yeast pedigree analysis. Accordingly, the present invention also provides methods of electrolessly depositing a ferromagnetic composition on the surface of a biologically-compatible substrate, such as a PDMS substrate. These methods include the steps of providing a biologically-compatible substrate comprising a surface; depositing an electroless deposition catalyst on the surface of the biologically-compatible substrate; and electrolessly depositing a ferromagnetic composition on the surface of the biologically-compatible substrate. Related methods include providing a substrate comprising a surface; exposing the surface to a plasma; optionally applying a sensitizer to the surface; depositing an electroless deposition catalyst on the surface; and electrolessly depositing a ferromagnetic composition on the surface of the substrate.

The present invention also provides methods for magnetically capturing one or more magnetically-labeled mother cells in a microfluidic device comprising one or more magnetic capture sites optically coupled to an imaging system, each of the magnetic capture sites fluidically coupled to one or more collection receptacles; detecting the generation of a daughter cell by at least one of the magnetically-labeled mother cells using the imaging system; and fluidically transporting the daughter cell from the microfluidic device to the one or more collection receptacles.

In additional aspects, the present invention provides methods that include providing a microfluidic device comprising one or more microchannels and one or more ferromagnetic structures disposed within the microfluidic device, at least one of the ferromagnetic structures located in proximity to one or more lumens of one or more of the microchannels; fluidically transporting a magnetically-labeled microorganism, cell, particle, or molecule through one of the lumens and towards one of the ferromagnetic structures; controllably magnetizing one of the ferromagnetic structures to create a magnetic field passing through a portion of one or more lumens; and controllably holding the magnetically-labeled microorganism, cell, particle, or molecule using the magnetic field within one of the lumens.

Other aspects of the present invention will be apparent to those skilled in the art in view of the detailed description and drawings of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1A is a micrograph of a budding yeast cell, mother cell on the left, daughter cell on the right; E. Schabtach et al., http://www.hhmi.org/genesweshare/a300.html.

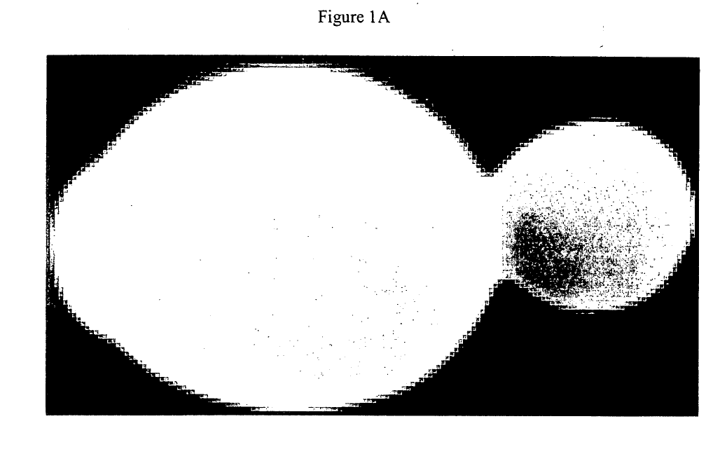

FIG. 1B is a photograph of an agar plate with the grown daughter colonies of Saccharomyces cerevisae (yeast), the final product of a yeast pedigree analysis.

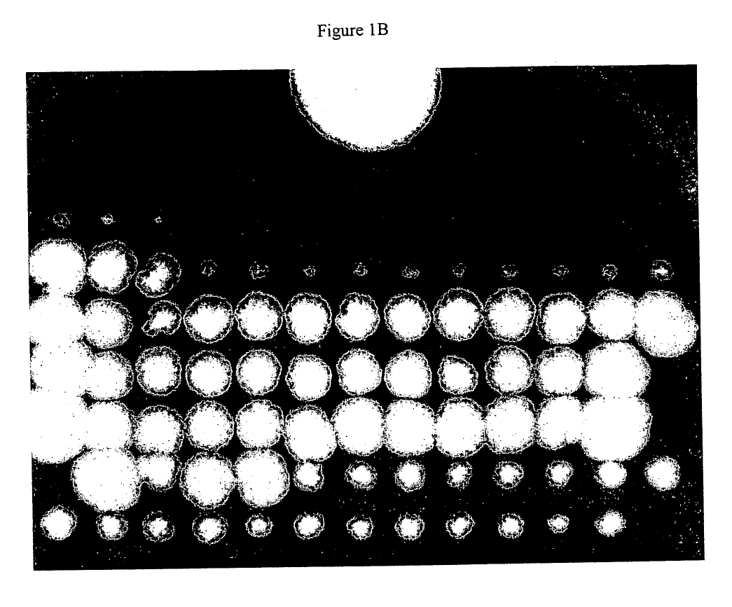

FIG. 2 is a photograph of an embodiment of the microfluidic device of the present invention residing adjacent to an optical system for imaging a magnetic capture site.

FIG. 3 is a schematic diagram of an embodiment of a microfluidic device having multiple magnetic capture sites; the input of each magnetic capture site can be selected between a microorganism, cell (e.g., a yeast cell), particle, or molecule, and a fluid source (e.g., nutrients); and the output of each magnetic cell capture site can be selected between waste and a collection receptacle (e.g., agar plate).

FIG. 4 schematically illustrates an embodiment of a microfluidic device of the present invention that has multiple magnetic trapping sites positioned in one microfluidic channel.

FIG. 5 is a photograph of a system of the present invention described in the examples.

FIG. 6 is a schematic illustration of two embodiments of the electroless deposition methods of the present invention for forming ferromagnetic structures on substrates; the deposition mask method is shown on the left and the etch mask method is shown on the right.

FIG. 7 is a schematic illustration of an embodiment of the electroless deposition methods of the present invention for forming ferromagnetic structures on substrates.

FIG. 8 is a photograph of auxiliary electrical equipment used in an embodiment of a system of the present invention described in the examples.

FIG. 9 is an illustration of an embodiment of a collection receptacle (agar reel) used in an embodiment of the system of the present invention.

FIG. 10 is a flowchart of a software program used in an embodiment of the system of the present invention.

FIG. 11 is a hardware schematic for an embodiment of an imaging system used in the present invention.

FIG. 12 is a photomicrograph of yeast cells as seen through an imaging bundle with fiber cores located 8 mm on center; the scale bar refers to the specimen plane.

FIG. 13 is a display screen of an embodiment of an image analysis system used in analyzing yeast images with user interface as seen through an imaging bundle with fiber cores located 2.21 mm on center; two budding (#1 and #3) and two non-budding yeast cells (#2 and #4) are accurately identified; the scale bar refers to the specimen plane.

FIG. 14 displays the image analysis results of yeast classification using the 2nd and 3rd invariant moments; the data points for nearly all budding yeast under both magnifications are located above or to the right of the thresholds.

FIG. 15 is a schematic illustration of an embodiment of a system of the present invention.

FIG. 16 is a photograph of an embodiment of a portion of a microfluidic device 1600 of the present invention.

FIG. 17 displays 4.5 μm paramagnetic beads captured in a microfluidic device using an embodiment of the system of the present invention; the microchannel wall is the white line running vertically through the frame; the permalloy wire tip is approximately 200 μm to the left of the channel wall.

FIG. 18 is a display of a user interface system of an embodiment of the system of the present invention that provides an image of a microfluidic channel and a yeast cell flowing through the lumen of the microfluidic channel.

FIG. 19 illustrates the operation of a pressure-actuated valve; the valve is normally open (A); to shut the valve, positive pressure is applied to the pneumatic layer to push the membrane against the ceiling of the fluid channel and close the channel (B).

FIG. 20 is a photomicrograph of Co—Ni—B ferromagnetic structures patterned on PDMS. The leftmost line is 20 μm wide.

FIG. 21 is a photomicrograph of a single captured yeast cell in an embodiment of a magnetic capture site of the present invention; the magnetic element is at the bottom of the image and the lower wall of the channel is the horizontal line across the image.

FIG. 22 is a photomicrograph of several ferromagnetic structures of the present invention having widths of 20 microns, 40 microns, 60 microns, 80 microns and 100 microns.

FIG. 23 is a still image from a yeast cell capture video using the system of the present invention; two yeast cells, each budding, approach from the left in the flow channel; the yeast cell, also budding, is held in place.

FIG. 24 is a schematic diagram of a process of fabricating ferromagnetic elements according to an embodiment of the present invention.

FIG. 25 depicts the results of plating height vs. time in plating bath for 27 samples; height was measured by atomic force microscopy (AFM); the height of the catalyst was measured by AFM at 30-40 nm (data not shown).

FIG. 26 depicts ferromagnetic elements after plating for 1 hour; A: an array of elements used to capture multiple cells (microfluidic channel not shown); B: 60× brightfield image of the straight extension at the end of one of the triangular tips.

FIGS. 27a-f are photomicrographs depicting single yeast cell capture using an embodiment of the present invention; the images were taken 1 second apart; the cells are attached to 50 nm paramagnetic beads, which are not visible in the image; the cell marked with a single arrowhead is captured at the tip of the ferromagnetic element in frame c; the cell marked with a double arrowhead flows freely; The two stationary marks above the tip appear to be optical debris.

FIG. 28 is a schematic illustration of an embodiment of a system of the present invention, for example, which can be used as an automated yeast pedigree analysis system.

FIG. 29 depicts a flow diagram for operating an embodiment of a system of the present invention, for example, which can be used for operating an automated yeast pedigree analysis system.

FIG. 30 is a schematic illustration of a simple single-cell capture device.

FIG. 31 is a photomicrograph depicting single-cell capture; the vertical line on the right is the wall of the microfluidic channel; this capture was performed without a protective layer between the magnet and the channel, which apparently resulted in slow etching of the magnet; protective layers can be provided between the magnet and the channel.

FIG. 32 diagrammatically illustrates two patterning methods that can be used for preparing magnetic capture sites; the deposition mask method is shown on the left and the etch mask method is shown on the right.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. When any variable occurs more than one time in any constituent or in any formula, its definition in each occurrence is independent of its definition at every other occurrence. Combinations of substituents and/or variables are permissible only if such combinations result in stable compounds.

As employed above and throughout the disclosure, the following terms, unless otherwise indicated, shall be understood to have the following meanings.

“Ferromagnetic” materials are strongly susceptible to magnetic fields and are capable of retaining magnetic properties when the field is removed. Ferromagnetism occurs when unpaired electrons in the material are contained in a crystalline lattice thus permitting coupling of the unpaired electrons.

The term “substrate” is used herein to refer to any suitable material that is capable of being microfabricated, e.g., silicon or silicon dioxide material such as quartz, fused silica or glass (borosilicates), polymeric materials, including engineering plastics, thermoplastics, UV or heat-cured resins, elastomers, and the like, carbon-based materials, and ceramics (including aluminum oxides and the like). One or more layers of material formed from a dimensionally stable support may form the substrate. Further, the substrate may comprise composite substrates such as laminates.

A “laminate” refers to a composite material formed from several different bonded layers of same or different materials. In the case of polymeric substrates, the substrate materials may be rigid, semi-rigid, or non-rigid, opaque, semi-opaque or transparent, depending upon the use for which they are intended. For example, devices that include an optical or visual detection element will generally be fabricated, at least in part, from transparent materials to allow, or at least facilitate that detection. Examples of particularly preferred polymeric materials include, e.g., polymethylmethacrylate (PMMA), polydimethylsiloxanes (PDMS), polyurethane, polyimide, polyvinylchloride (PVC), polystyrene, polysulfone, polycarbonate, and the like. Preferably, these materials will be phenolic resins, epoxies, polyesters, thermoplastic materials, polysulfones, or polyimides and/or mixtures thereof.

In addition to constructing the substrate using conventional printed circuit board composites, alternative structures can be used. For example, for certain applications the use of plastic films, metals, glasses, ceramics, injection molded plastics, elastomeric layers, ferromagnetic layers, sacrificial photo resist layers, shaped memory metal layers, optic guiding layers, polymer based light displays or other suitable materials may be used. These may be bonded to the substrate to form the system with or without an adhesive bonding layer.

The term “microfluidic” refers to a system or device having a network of chambers connected by channels, tubes or other interconnects in which the channels may act as conduits for fluids or gasses. Microfluidic systems are particularly well adapted for analyzing small sample sizes. Sample sizes are typically are on the order of nanoliters and even picoliters. Similar apparatus and methods of fabricating microfluidic devices are also taught and disclosed in U.S. Pat. Nos. 5,858,195, 5,126,022, 4,891,120, 4,908,112, 5,750,015, 5,580,523, 5,571,410, and 5,885,470, incorporated herein by reference.

“Microfluidic analytical systems” refer to systems for forming chemical, clinical, or environmental analysis of chemical and/or biological specimens. Such microfluidic systems are generally based on a chip. These chips are preferably based on a substrate for micromechanical systems. Substrates are generally fabricated using photolithography, wet chemical etching and other techniques similar to those employed in the semiconductor industry. Microfluidic systems generally provide for flow control and physical interactions between the samples and the supporting analytical structure. The microfluidic device generally provides conduits and chambers arranged to perform numerous specific analytical operations including mixing, dispensing, valving, reactions, detections, electrophoresis and the like.

Microfluidic devices can be fabricated out of any material that has the necessary characteristics of chemical compatibility and mechanical strength. One exemplary material is silicon since a wide range of advanced microfabrication and micromachining techniques have been developed for it and are well known in the art. Additionally, microfluidic devices can be produced directly in electrically insulating materials. The most widely used processes include isotropic wet chemical etching of glass or silica and molding of plastics. The channels are typically defined by photolithographic techniques and etching away the material from around the channel walls produces a freestanding thin walled channel structure. Freestanding structures can be made to have very thin or very thick walls in relation to the channel width and height. The walls, as well as the top and bottom of a channel can all be of different thickness and can be made of the same material or of different materials or a combination of materials such as a combination of glass, silicon, and a biologically-compatible material such as PDMS. Sealed channels or chambers can be made entirely from biologically-compatible material such as PDMS.

The systems of the present invention generally include a microfluidic device having one or more magnetic capture sites optically coupled to an imaging system, wherein the magnetic capture sites are each capable of magnetically capturing a magnetically-labeled mother cell; each of the magnetic capture sites fluidically coupled to one or more collection receptacles; wherein the imaging system is capable of detecting the generation of a daughter cell by the magnetically-labeled mother cell and actuating the microfluidic device to fluidically transport the daughter cell to the one or more collection receptacles. The microfluidic device typically includes one or more microfluidic structures, such as microchannels, for transporting, capturing, imaging and separating cells. Suitable microfluidic devices are typically composed of one or two substrates. The microchannels can be formed by using any of the known microchannel patterning techniques. Suitable microchannel patterning techniques include the steps of applying a photoresist to a glass or silicon substrate, lithographically patterning a positive or negative photoresist on the glass or silicon substrate, developing the photoresist, to provide a microfluidic surface relief template, coating or applying a suitable biologically-compatible material to the microfluidic surface relief template, curing or hardening the biologically-compatible material on the microfluidic surface relief template, and removing the biologically-compatible material to provide a biologically-compatible substrate having microfluidic structures patterned in its surface. Patterned biologically-compatible substrates are typically fluidically sealed to form microfluidic structures through which fluids can be transported therethrough. Examples of fluidically sealing the microfluidic structures include disposing a covering material, film, or second substrate adjacent to the biologically-compatible substrate surface that contains the microfluidic structures.

Suitable biologically-compatible substrates include a variety of substrate materials that can include a polymer, ceramic, glass, metal, alloy, or any combination thereof. In some embodiments, the polymer comprises a biologically-compatible polymer. Suitable biologically-compatible polymers include a plurality of units derived from a siloxane, an alkyl oxide such as ethylene oxide, an acrylic, an amide, a polymerizable carboxylic acid group, or any combination thereof. When the biologically-compatible polymers include a plurality of units derived from a siloxane, the siloxane units typically include a plurality of monomers that include dimethyl siloxane, or any combination thereof. A preferred biologically-compatible polymer composed of a plurality of siloxane units is polydimethyl siloxane (“PDMS”). Any other type of polymeric material that can be fabricated into optically transparent microfluidic devices, for example polymethylmethacrylate (“PMMA”), can also be used.

Substrate materials that are not necessarily biologically-compatible can also be used in some embodiments of the present invention. In these embodiments, substrate materials that are not alone biologically-compatible can be made compatible using a suitable surface treatment or coating to make them biologically-compatible. Suitable surface treatments or coatings can include a thin film of a biologically-compatible material applied to the surface of a typically biologically-incompatible substrate. For example, the microfluidic structures patterned in a biologically-incompatible substrate can be surface treated with an optional adhesion modifying agent and then coated with a thin film of a biologically-compatible material, such as PDMS. In this embodiment, cells flowing through the lumens of a PDMS-coated microfluidic structure even though the substrate is composed of a biologically-incompatible material. As used herein, biologically-incompatible materials are typically defined as those materials that gives rise to adhesion of the cells to a surface of the material or at an interface in proximity to the material. As used herein, biologically-compatible materials are typically defined as those materials that do not give rise to an interaction of cells to a surface of the material are at an interface in proximity to the material. Typical biologically-incompatible surfaces include many hydrophobic plastics, many glasses, and many metals.

Suitable microfluidic devices can have a wide ranging number of magnetic captures sites, for example, from one to about 1,000 magnetic capture sites. In certain preferred embodiments the microfluidic devices can have up to about 100 magnetic capture sites, and in other embodiments the microfluidic devices can have up to about 10 magnetic capture sites. Each capture site may have one or more magnetic elements (i.e., ferromagnetic structures) for capturing one or more magnetically-labeled cells. The number of magnetic elements per capture site can be in the range of from 1 to about 100, or in the range of from about 2 to about 50, or in the range of from about 4 to 25, or in the range of from about 8 to 12. Accordingly, a wide range in number of cells can be analyzed using one microfluidic device. For example, from 1 to about 100,000 cells, typically from 100 to 50,000 cells, and more typically from about 500 to 10,000 cells can be readily analyzed using the systems of the present invention on a 2 inch by 3 inch substrate platform. Even higher numbers of cells can be readily analyzed by increasing the scale of the substrate platform, for example, doubling the linear dimensions will give rise to a quadrupling of the area and the number of cells that can be analyzed.

As used herein, a magnetic capture site is the region within a microfluidic device where the end of a tapered region of a ferromagnetic structure is proximate to a lumen of a microchannel for magnetically capturing and releasing a magnetically-labeled microorganism, cell, particle, molecule, or any combination thereof. The ferromagnetic structures are each composed of a ferromagnetic composition. Suitable ferromagnetic compositions comprise iron, cobalt, nickel, boron, or any combination thereof, for example, cobalt-nickel-boron, iron-nickel-boron, iron-cobalt-nickel-boron, iron-cobalt-boron, nickel-boron, cobalt-boron, iron-boron, or any combination thereof. Other materials having a high magnetic permeability can also be incorporated in the ferromagnetic compositions. The ferromagnetic compositions can be deposited onto a biologically-compatible substrate, such as PDMS, using any one of the known deposition techniques for depositing magnetic materials on polymers. Preferably, an electroless deposition process is used for depositing ferromagnetic materials on a biologically-compatible substrate as further described herein.

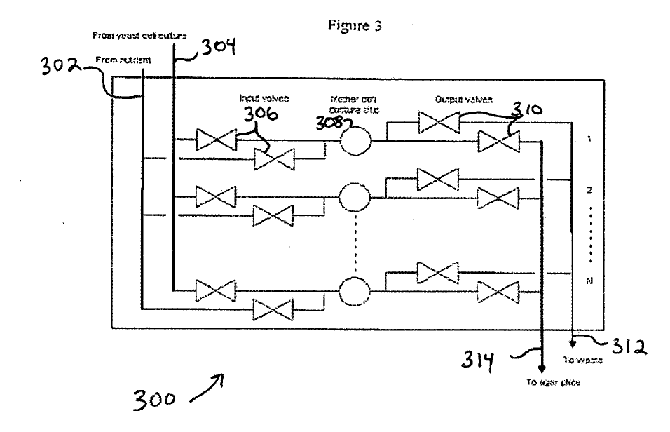

The ferromagnetic structures can be disposed on or within the biologically-compatible substrate, and typically at least a portion of the ferromagnetic structures are disposed on or within the biologically-compatible substrate. The ferromagnetic structures are suitably provided as a ferromagnetic element or material capable of creating a localized electromagnetic field across the direction of flow through a lumen of a microchannel in the microfluidic device to form a magnetic capture site. A magnetic capture site can be located as the region within the lumen where a magnetic field gradient is induced using a ferromagnetic structure composed of a ferromagnetic compound that comprises a point-like or tapered shape proximate to the lumen of a microchannel. For example, a point-like shaped ferromagnetic structure is suitably provided as a point, line end, dot, corner of a polygon such as a triangle, square, parallelogram or other polygonal shape, edge or line or polygon, or any combination thereof. The end of a tapered ferromagnetic structure that is located proximate to the lumen of a microchannel is also capable of providing a magnetic field gradient in the magnetic capture site. The ends of tapered magnetic structures may terminate as a point, line or arc. The magnetic fields of the ferromagnetic structures are suitably magnetized using a magnetic field source located external to the magnetic capture site. For example, a permanent magnet, an electromagnet, or any combination thereof can be used to magnetize the magnetic element (i.e., the ferromagnetic structure) to give rise to a magnetic field emanating from the point-shaped portion in proximity to the lumen for capturing magnetically-labeled cells. In additional embodiments, the permanent magnet or electromagnet can be a microfabricated structure disposed on or within the microfluidic device. For example, microfabricated electromagnets for use in microfluidic devices are disclosed in paragraphs [0062] to [0063] and FIG. 3 of U.S. Patent Application Publication No. 2003/0129646, filed Jul. 10, 2003, to Briscoe et al., the identified portion of which is incorporated by reference herein. The ferromagnetic element can also be prepared from the core of these electromagnets.

The magnetic elements or ferromagnetic structures can be disposed on a biologically-compatible substrate proximate to one or more microchannels comprising a lumen. A suitable ferromagnetic composition can be provided as a lithographic pattern on the biologically-compatible substrate. For example, the lithographic pattern can be provided by photolithography, imprint lithography, or any combination thereof. A suitable lithographic pattern typically comprises a plurality of regions having a dimension in the range of from about 500 nm to about 1000 microns, typically from about 1 micron to about 500 microns, more typically from about 2 microns to about 250 microns, and even more typically from about 5 microns to about 100 microns. In various embodiments, the plurality of regions are typically separated by a distance in the range of from about 500 nm to about 5000 microns, more typically in the range of from about 1 micron to about 2000 microns, even more typically in the range of from about 2 microns to about 1000 microns, further typically in the range of from about 5 microns to about 500 microns, and even further typically in the range of from about 10 microns to about 200 microns. The microfluidic devices used in the systems of the present invention can be composed of one or more substrates. In embodiments composed of at least two substrate, the substrates can be fluidically sealed to each. For example, the microfluidic devices can be composed of a first substrate fluidically sealed to a second substrate. In certain embodiments, for example, the first substrate comprises the ferromagnetic structures disposed on or within a biologically-compatible substrate, and the second substrate comprises one or more microfluidic structures. Suitable microfluidic structures on a substrate typically have a dimension in the range from about 1 micron to about 1000 microns for transporting and analyzing a variety of fluids and fluid components, including biological materials such as cells or DNA, nutrients, reagents, biological products, and wastes.

The first substrate and the second substrate can be fluidically sealed to give rise to microchannels having lumens and other fluidically sealed structures residing in the interfacial region between the two substrates. The sealed substrates typically give rise to at least one of the magnetic capture sites being in fluidic communication with one or more of the microfluidic structures. In one embodiment, the second substrate comprises one or more microfluidic structures each having a dimension in the range from about 1 micron to about 1000 microns. In another embodiment, the first substrate comprises one or more microfluidic structures each having a dimension in the range from about 1 micron to about 1000 microns. In still another embodiment, the ferromagnetic elements are disposed on top of or adjacent to the one or more microfluidic structures disposed on the first substrate, the second substrate, or both. Suitable microfluidic structures include microchannels, micropumps, microvalves, microposts, micromixers, microsensors, microtransducers, or any combination thereof.

Preferred embodiments incorporate at least one of the magnetic capture sites being in fluidic communication with one or more microchannels. Accordingly, in certain embodiments, at least a portion of the plurality of magnetic capture sites are each in fluidic communication with at least one input microchannel and at least one output microchannel. Input microchannels can be suitably used, for example, to be in fluidic communication with a cell culture source and a nutrient source. Output microchannels can be suitably used, for example, to be in fluidic communication with a waste receptacle and one or more collection receptacles. Suitable waste receptacles include a tube, vial, dish, bottle or drain for disposing of unwanted fluids and biological material. For controlling the flow of mother cells, daughter cells, nutrients, wastes, or any combination thereof through the microfluidic device, at least one of the input and output microchannels typically comprise at least one microfluidic valve. Suitable microfluidic valves include a pneumatic microfluidic valve, a magnetic microfluidic valve, an electrostatic microfluidic valve, or any combination thereof.

Various embodiments of the present invention having input and output microchannels may also include at least one input microchannel being in fluidic communication with at least one pump. Suitable pumps include peristaltic pumps and positive displacement pumps, both types of which are commercially available. The microfluidic devices having input and output microchannels may also include at least one output microchannel is in fluidic communication with at least one vacuum source. Suitable vacuum sources include free-standing vacuum pumps as well as house vacuum sources. Pumps and vacuum sources are typically provided to assist the transport of fluids within the lumens of the microchannels in the microfluidic devices. Pumps and vacuum sources can also be provided to control the operating pressure within the microfluidic device.

The imaging systems used in the systems of the present invention can be suitably provided using a variety of optical components. Suitable optical components include at least one optical lens, at least one optical fiber imaging bundle, at least one optical fiber, at least one optical detector, at least one image processor, at least one optical filter, at least one mirror, at least one light source, or any combination thereof. In the imaging systems of the present invention, an optical fiber imaging bundle is used and positioned among the components of the system to optically transmit an image of at least one of the magnetic capture sites to a suitable image processor. The image processor receives the image of the magnetic capture site using a suitable detector, such as a charge-coupled device or digital camera to create a digital image of the magnetic capture site. As provided further below, an image processor operates on the digital image of the magnetic capture site to determine, for example, the capture of cells and subsequent cellular events, such as budding. Accordingly, one imaging system embodiment comprises an optical train for transmitting at least one image of the at least one magnetic capture sites to an image processor. Suitable image processors used in the present invention include a processor and an image processing algorithm for ascertain cellular events within a magnetic capture site. As the magnetic capture sites are capable of having multiple magnetic elements or ferroelectric structures for capturing, holding, releasing, or any combination thereof, magnetically-labeled cells, the imaging systems provided herein are capable of analyzing multiple cells, simultaneously, sequentially, or any combination thereof. For example, a digital image of one of the magnetic capture sites may include from 1 to about 100 cells, or from about 2 to about 50 cells, or from about 4 to about 25 cells, or from about 8 to about 12 cells. Accordingly, a wide range in number of cells located at a magnetic capture site can be analyzed using the image processor.

Suitable imaging systems used in several embodiments of the present invention typically include at least one light source positioned to illuminate the one or more magnetic capture sites, an optical train for transmitting one or more images of the magnetic capture site to an optical detector, the optical detector capable of communicating the image to an image processor. In these embodiments, a magnetic capture site can be situated between the light source and the optical train.

Two substrates can be fluidically sealed to each other in certain cases to give rise to microfluidic devices that can be optically transparent. The degree of optical transparency needed can be that amount that is capable of transmitting an image of the one or more magnetic capture sites through the first substrate, the second substrate, or both. The magnetic capture site can be within the field of view of the optical train. Accordingly, a suitable orientation of the light source, microfluidic device, and optical train provides an image of the magnetic capture site that is optically transmitted to the imaging system. In alternate embodiments, using epifluorescent optical systems, the optical train also transmits the light source, so the magnetic capture site is positioned within the field of view of the optical train.

Various optical elements may be spatially fixed within the system, or they may be spatially translatable. For example, an optical lens portion of the optical train may be spatially positioned using a suitable robotic arm or servo motor device to image one or more of the magnetic capture sites. An optical fiber bundle having from about 100 to about 100,000 fiber cores having a diameter in the range of from about 1 μm to about 10 μm can be part of the optical training for coupling the spatially-positionable optical lens portion of the optical lens to a fixed portion of the imaging processor comprising the CCD or digital camera, as well as the data processor. Accordingly, in certain embodiments a portion of the imaging system is capable of being spatially translated to individually receive an image of more than one of the magnetic capture sites, and then transmitting the optical image through an optical fiber bundle to a spatially-fixed image processor.

Systems may additionally include one or more spatial translation devices for spatially positioning the microfluidic device, at least a portion of the imaging system for receiving an image of the one or more magnetic capture sites, or any combination thereof.

Suitable imaging systems can be capable of filtering out noise introduced by the fiber optical bundle. For example, the imaging system can filter out noise introduced by the fiber optical bundle by: capturing a gray-scale image of one or more magnetic capture sites; smoothing the gray-scale image; converting the smoothed gray-scale image to a binary image using a locally generated threshold based on the mean and standard deviation of the subimage; and segmenting the binary image into regions of pixels.

The imaging system can be capable of detecting the division progress of the mother cell and the generation of the daughter cell. For example, the imaging system can detect the division progress of the mother cell and the generation of the daughter cell by the following steps: capturing a gray-scale image of one or more capture sites; calculating the area of each region of pixels that is a potential cell; removing the regions of pixels smaller than the area of a mother cell from the binary image; and classifying each remaining region of pixels as being composed of a mother cell or of a mother cell and a daughter cell.

The present invention also provides methods of electrolessly depositing a ferromagnetic composition on the surface of a substrate. Suitable substrates include glass, plastic, silicon, polymer, ceramic and any combinations thereof. In preferred embodiments the substrates are composed of biologically-compatible materials, biologically-compatible surfaces, or any combination thereof. These methods include the steps of providing a substrate comprising a surface; depositing an electroless deposition catalyst on the surface of the substrate; and electrolessly depositing a ferromagnetic composition on the surface of the substrate. Suitable substrates include any of the substrates as described above for the various microfluidic devices, and preferably the substrate comprises a siloxane material such as PDMS. In addition to the steps of electrolessly depositing a ferromagnetic composition on the substrate, one embodiment of the process may further include the step of lithographically patterning the ferromagnetic composition on the surface of the substrate. Lithographically patterning the ferromagnetic composition is desirably carried out, for example, to create ferromagnetic structures and various magnetic elements on the surface of biologically-compatible substrate, such as PDMS. Suitable techniques for lithographically patterning the ferromagnetic composition are well known in the art and include at least one of photolithography, imprint lithography, or any combination thereof.

The step of depositing an electroless deposition catalyst on the surface may also include transferring catalyst from a second compliant substrate surface to the surface. Suitable compliant substrates include a foam or film material, for example a vinyl foam. In one example, ferromagnetic composition can be deposited as follows: place a PDMS stamp (e.g., mask) on a substrate and plasma treat for 10 min; remove mask; wet substrate; wet a second compliant substrate and apply catalyst particles (e.g., ground dry catalyst, such as palladium chloride) onto the wet second compliant substrate surface, and transfer the catalyst particles onto the surface of the substrate, for example, by rubbing the catalyst particles on the second compliant substrate onto the substrate (e.g., for about 10 seconds). The catalyst sticks mainly to the portion of the substrate that was shielded from the plasma using the mask. The substrate is then rinsed, the excess catalyst particles are removed, and a suitable ferromagnetic composition is electrolessly deposited on the surface of the substrate (e.g., substrate is then placed into a plating bath containing a suitable solution for electroless deposition of the ferromagnetic composition).

Magnetic elements and ferromagnetic structures deposited on biologically-compatible substrates can be used in a variety of applications involving biological material analysis instrumentation as well as medical devices. The ferromagnetic structures prepared using the electroless deposition methods described herein are useful for preparing magnetic capture sites that are capable of controlling the motion and location of magnetically-labeled cells in microfluidic devices. For example, in one embodiment, the present invention provides a method comprising the steps of magnetically capturing one or more magnetically-labeled mother cells in a microfluidic device comprising one or more magnetic capture sites optically coupled to an imaging system, each of the magnetic capture sites fluidically coupled to one or more collection receptacles; detecting the generation of a daughter cell by at least one of the magnetically-labeled mother cells using the imaging system; and fluidically transporting the daughter cell from the microfluidic device to the one or more collection receptacles. Accordingly, in this and other embodiments, the one or more magnetic captures sites can be provided by the steps of electrolessly depositing a ferromagnetic composition on the surface of a biologically-compatible substrate, such as PDMS, as provided herein. Suitable imaging systems for use in these embodiments, for example, by use of an optical train and image processor, are further described herein.

Methods of controllably holding magnetically-labeled cells in a microfluidic device are also provided. These methods include providing a microfluidic device comprising one or more microchannels and one or more ferromagnetic structures disposed within the microfluidic device, at least one of the ferromagnetic structures located in proximity to one or more lumens of one or more of the microchannels; fluidically transporting a magnetically-labeled microorganism, cell, particle, or molecule through one of the lumens and towards one of the ferromagnetic structures; controllably magnetizing one of the ferromagnetic structures to create a magnetic field passing through a portion of one or more lumens; and controllably holding the magnetically-labeled microorganism, cell, particle, or molecule using the magnetic field within one of the lumens. Controlling the magnetization of the ferromagnetic structures and providing ferromagnetic structures proximately located to the lumens gives rise to the magnetic capture sites that are capable of controlling the spatial position, velocity and orientation of magnetically-labeled microorganisms, cells, particles and molecules.

Suitable magnetically-labeled microorganism includes a magnetically-labeled yeast, a magnetically-labeled bacteria or a magnetically-labeled virus. Suitable magnetically-labeled particles include magnetically-labeled vesicles, magnetically-labeled liposomes, magnetically-labeled macromolecular complexes, or any combination thereof. Suitable magnetically-labeled molecules include a magnetically-labeled nucleic acids, magnetically-labeled amino acids, magnetically-labeled carbohydrates, or any fragment or combination thereof. Suitable magnetically-labeled nucleic acids include DNA, RNA, or any fragment or combination thereof. Suitable magnetically-labeled amino acids includes proteins, prions or any fragment or combination thereof.

The ferromagnetic structures can be located in any of a number fashions relative to the lumens of the microchannels. In one embodiment the ferromagnetic structures are located exterior to the lumen of one or more of the microchannels. In another embodiment, the ferromagnetic structures are located completely exterior to the lumen of one of the microchannels. Other embodiments provide that the ferromagnetic structures are located partially exterior to one or more lumens of one of the microchannels. The ferromagnetic structure comprises a point or a narrow rectangle comprising one or more point-like tips (e.g., a pencil-point type shape), a triangle or any combination thereof, in proximity to a lumen of one of the microchannels. Without being bound by any theory of operation, it is believed that the point-like tips create a large magnetic field gradient in a narrow region within the lumen to trap a magnetically-labeled microorganism, cell, particle or molecule. For example, a magnetic field can be applied by an external magnetic field source (e.g., a permanent magnet or an electromagnet) to a portion of the ferromagnetic structure that is located away from the tip. The ferromagnetic structure becomes magnetized by the externally applied magnetic field source, and the resulting magnetic field exiting the ferromagnetic structure falls off sharply adjacent to the tip to create a large magnetic field gradient.

The ferromagnetic structures can be oriented in any of a number of fashions with respect to the lumens of the channels for the purposes of providing a localized magnetic field in the lumens of various microstructures within a suitable microfluidic device. In one embodiment, for example, a plurality of ferromagnetic structures can disposed along one or more of the microchannels. The ferromagnetic structures are typically disposed exterior to the microchannels with the tip portion of the ferromagnetic structures being located proximate to microchannels to provide a localized magnetic field within the lumen of the microchannel.

The lumen can be in fluid communication with an inlet and an outlet of the microchannel. Here, a magnetically-labeled microorganism, cell, particle, or molecule and a fluid medium flows into the lumen through the inlet, the magnetically-labeled microorganism, cell, particle, or molecule is controllably held using the magnetic field in the lumen, and the fluid medium flows out of the outlet. This method is suitably used, for example, to provide fluid media that comprise nutrients for magnetically-labeled cells and organisms. In one embodiment, the fluid medium flows past the magnetically-labeled microorganism, cell, particle, or molecule through the lumen while the magnetically-labeled microorganism, cell, particle, or molecule is controllably held. The fluid flows past the magnetically-labeled microorganism or cell is controllably held by the localized magnetic field within the lumen as one or more daughter cells are produced. The one or more daughter cells can be monitored by the image processing system and fluidically transported to one or more collection receptacles for subsequent genetic analysis. Suitable collection receptacles include an agar plate, a vial, a test tube, a multiwell plate, a molded agar receptacle, or any combination thereof.

Magnetically-labeled microorganisms or cells suitably include one or more microorganisms or cells and a plurality of paramagnetic or superparamagentic particles linked to the microorganism or cell. In one embodiment, at least a portion of the paramagnetic or superparamagnetic particles are linked to an exterior surface of the microorganism or cell. In other embodiments at least half of the plurality of paramagnetic or superparamagnetic particles have a particle size in the range of from about 5 nm to about 50 microns. At least half of the plurality of paramagnetic or superparamagnetic particles typically have a diameter in the range of from about 5 nm to about 50 microns. More typically, the plurality of paramagnetic or superparamagnetic particles have an average particle size in the range of from about 5 nm to about 50 microns, and even more typically, the plurality of paramagnetic or superparamagnetic particles have an average particle size in the range of from about 10 nm to about 10 microns, or 20 nm to 5 microns, or 50 nm to about 250 nm. Many suitable paramagnetic or superparamagnetic particles are commercially available in sizes less than 1 micron.

Suitable microorganisms that can be magnetically labeled include Saccharomyces cerevisiae, S. uvarum, S. exiguus, S. servazzii, S. kluyveri, Schizosaccahromyces pombe, Zygosaccharomyces rouxii, Debaryomyces hansenii, Pichia pastoris, Candida albicans, C. galbratus, C. tropicalis, Yarrowia lipolytica, Cryptococcus neoformans, Kluyveromyces lactis, Ashbya gossypii, Escherichia coli, Caulobacter crescentus, or any combination thereof.

Suitable cells that can be magnetically labeled include eukaryotic cells and prokaryotic cells. Suitable eukaryotic cells include mammalian cells, such as human cells, murine cells, porcine cells, bovine cells and primate cells. Any type of human cells can be magnetically labeled and analyzed using the device and methods of the present invention, examples of which include red blood cells, white blood cells, stem cells, skin cells, cheek cells, sperm cells, egg cells, fertilized egg cells, bone cells, muscle cells, nerve cells, cells from any internal organ, as well as any healthy, diseased, mutant, precancerous or cancerous version thereof, or any combination thereof. Suitable prokaryotic cells include bacteria, cyanobacteria, or any combination thereof.

Suitable magnetic labeling compounds include paramagnetic and superparamagnetic particles (beads). Suitable magnetic labeling compounds and methodologies are known in the art, many of which are commercially available from companies such as Dynal Biotech (Norway), EMD Biosciences, Miltenyi Biotec Inc., Pierce Biotechnology and Quantum Magentics. For example, Dynal Biotech, Norway (www.dynal.no); provides Dynabeads™ having monodispersed spherical particles in the range of from about 1 micron to about 5 microns in diameter; Miltenyi Biotec Inc., Germany (www.miltenyibiotec.com) provides antigen-linked 0.05 micron (50 nm) magnetic beads; and Quantum Magnetics, Sweden (http://www.intermedica.se/quantum.html) provides 50 nm and 250 nm magnetic beads.

Suitable methods of labeling microorganisms with magnetic labeling compounds include direct attachment of magnetic beads coupled to molecules that bind specific factors naturally expressed on the microbe surface, and indirect attachment of magnetic beads coupled to molecules that bind additional factors experimentally conjugated to the microbe surface. Suitable factors naturally expressed on the surface of microbes include surface proteins, the sugar/starch moieties of modified surface proteins, and lipids.

Suitable methods of labeling cells with magnetic labeling compounds include direct attachment of magnetic beads coupled to molecules that bind specific factors naturally expressed on the cell surface, and indirect attachment of magnetic beads coupled to molecules that bind additional factors experimentally conjugated to the cell surface. Suitable factors naturally expressed on the surface of cells include surface proteins, the sugar/starch moieties of modified surface proteins, and lipids.

When one or more daughter cells are produced, the methods may further comprise one or more steps for magnetically labeling at least one of the daughter cells. Daughter cells can be magnetically labeled by use of a microfluidic mixing chamber on a microfluidic device that combines the daughter cells with magnetic particles with suitable linking compounds. The daughter cells may also be removed from the microfluidic device and magnetically labeled following any of the procedures as described herein. The methods of the invention may also be suitably carried out be repeating the following steps sequentially one or more times: optionally magnetically-labeling a daughter cell, for example, by use of a microfluidic mixing chamber; fluidically transporting the magnetically-labeled daughter cell through the lumen and towards a second magnetic structure; controllably magnetizing the second magnetic structure to create a second magnetic field; and controllably holding the magnetically-labeled daughter cell with the second magnetic field. In these embodiments, the second magnetic structures are located down stream from the ferromagnetic structure where the daughter cell was produced. In some kinds of cells (e.g., bacteria), the daughter cell may receive some of the mother's paramagnetic or superparamagnetic particles. In these embodiment, the mother cell can received additional magnetically labeling as it is held in the magnetic capture site. For example, suitable paramagnetic or superparamagnetic particles are fluidically transported into the magnetic capture site as the mother cell is held.

One or more steps of imaging the magnetically-labeled microorganism, cell, particle or molecule can also be provided. The imaging steps are typically carried out to monitor the magnetically-labeled microorganism, cell, particle or molecule for subsequent analysis. Accordingly, the magnetically-labeled microorganism, cell, particle or molecule is imaged as it is being controllably held by the magnetic field within the lumen. After imaging, for example, the production of a daughter cell by the cell can be determined by digitizing the images and analyzing the digitized images with a suitable image processor as described further herein.

The one or more ferromagnetic structures can be controllably magnetized using a permanent magnet or an electromagnet. Magnetization control can be carried out manually. In preferred embodiments, the step of controllably magnetizing the ferromagnetic structure is controlled by a central processing unit. For example, a central processing unit can be provided that receives a data input from an image processing algorithm that signifies the production of a daughter cell by a mother cell, or by the inactivity (i.e., death) of the mother cell. The central processing unit then sends signals to control the magnetization of the ferromagnetic structures to control the holding and releasing of the magnetically-labeled cells. The central processing unit also communicates with a fluid control processor for controlling the flow of microorganisms, cells, particles, molecules, fluids, or any combination thereof. For example, continuous inactivity of the mother cell may be detected by the image processor and communicated to the central processing unit (CPU). The CPU sends a signal to a magnetic structure magnetization controller that the cell should be released. The corresponding magnetic structure is demagnetized, the inactive microorganism or cell is released, and the fluid controller processor controls the fluid pumping mechanisms and microvalves to direct the inactive cell to a suitable collection receptacle or waste stream.

Accordingly, the methods of the present invention provide for the additional step of controllably releasing the magnetically-labeled cell from the magnetic field. The released cell may be controllably held by a second ferromagnetic structure downstream from the ferromagnetic structure. In this embodiment, the released cell can be fluidically transported to a second microchannel, an agar plate, a test tube, a vial, a multiwell plate, a microreactor, a valve, an imaging area, or any combination thereof.

Additional magnetic structures and microfluidic structures can also be provided for receiving magnetically released microorganisms, cells, particles or molecules. For example, daughter cells can be controllably fluidically transported to a second magnetic capture site or a second microfluidic channel for subsequent analysis. Controlled fluidic transportation can be carried out by the following steps: the image processor detects the production of a daughter cell by a mother cell and sends a signal of this event to the CPU; the CPU sends a signal to a fluid control processor which adjusts the flow parameters (e.g., the flow rates, operating pressures and microvalve positions on the microfluidic device); the daughter cell is fluidically directed to an optional microfluidic mixing chamber where the daughter cells can be optionally magnetically labeled; and one ore more microfluidic valves are adjusted to direct the optionally magnetically-labeled daughter cell to a second magnetic capture site, a second microfluidic channel, or another microstructure.

The ferromagnetic structures may be located in any of a variety of positions within or on the microfluidic device. In one embodiment, a ferromagnetic structure is located completely within the lumen of one or more of the microchannels. The ferromagnetic structures can be magnetized using a permanent magnet or an electromagnet that directly contacts or is in close proximity to the ferromagnetic structures. When the permanent magnet or electromagnet does not directly contact the one or more ferromagnetic structures, its proximity to the ferromagnetic structure is sufficient to effect magnetization of the ferromagnetic structure. Sufficient magnetization of the ferromagnetic structure is provided when a magnetic field of strength sufficient emanates from the tip to capture a magnetically-labeled microorganism, cell, particle or molecule within a magnetic capture site.

One or more of the ferromagnetic structures can be each capable of creating a magnetic field passing through a portion of the lumens of two separate microchannels. For example, a dual-tipped ferromagnetic structure can be provided having a single magnetization body that bifurcates into two tips, each tip of which provides a magnetic field for separate lumens of separate microchannels.

Microfluidic devices having a wide range in number of microfluidic structures and ferromagnetic structures for providing a wide range in number of magnetic capture sites for magnetically-labeled microorganisms, cells, particles and molecules can also be used in the methods of the present invention. For example, microfluidic devices of various sizes and configurations can be provided having up to about 10,000, or up to about 1,000, or up to about 100, or up to about 20 ferromagnetic structures being disposed in proximity to one or more microchannels. Typically each of the ferromagnetic structures are disposed in proximity to one of the microchannels to provide one or more magnetic capture sites.

The microfabricated magnetic capture sites as described herein also have a variety of uses in microfluidic and analytical instruments. For example, the magnetic capture sites as provided herein can be used to magnetically couple any type of entity in solution that has some kind of magnetic label or handle. This magnetic coupling can be used to control the spatial position, velocity, orientation, or any combination thereof, of one or more magnetically coupled entities. For example, the microfabricated magnetic capture sites of the present invention can be used to hold a magnetically-labeled entity in place within a lumen of a microfabricated structure as fluid medium flows past the magnetically-held entity. This magnetically holding, for example, can be used to purify the entity or concentrate the entity from other components in solution with the entity. Examples of entities that can be magnetically labeled and spatially controlled in this fashion include microorganisms, cells, particles and molecules. In another example, the magnetic capture sites can be used to synthesize a variety of molecules and macromolecular complexes. Examples of molecules that can be magnetically captured include nucleic acids (e.g., DNA and RNA) and amino acids (e.g., proteins). Examples of macromolecular complexes include any of a variety of combinations and assemblies (e.g., self-assembled) of atoms and molecules. Exemplary macromolecular constructs containing both protein and DNA.

The patterning of individually addressable ferromagnetic structures can be implemented in a microfluidic device, or it may be done on top of a suitable substrate (e.g., composed of silicon, glass, plastic, an elastomer, or any combination thereof). In one embodiment, magnetic labeling is used to immobilize arrays of cells or molecules on a substrate, and the substrate is then dipped into a macroscopic vat, or a series thereof, to expose at least a portion of the cells to a substance, or to expose the magnetically-labeled entities to a subsequent solution in a serial synthesis reaction. Serial microfluidic DNA synthesis can also be carried out using the microfluidic channels, for example, flow base “C” followed by base “A”, and so on.

The systems, components and devices of the present invention can also be used for monitoring stem cells through successive divisions. Stem cells can be isolated from mixed populations by labeling with fluorescent antibodies that attach to stem cell-specific cell surface markers (proteins), and then using fluorescence activated cell sorting (FACS) to purify the fluorescent cells. In one embodiment, a stem cell is isolated using the devices and methods of the present invention. Stem cells are suitably magnetically labeled using magnetic beads conjugated to the stem cell-specific surface markers (i.e., protein antibodies). The magnetically-labeled stem cells are magnetically held in the magnetic captures sites of the present inventions. The stem cell continues to magnetically “hold on” through multiple cell divisions in the magnetic capture site. The non-stem cell progeny of the magnetically-held stem cell does not remain magnetically held in the magnetic capture site of the present invention. Without being bound by any particular theory of operation, the non-stem cell progeny of the stem cell do not remain magnetically held in the magnetic capture site because the progeny do not express the cell surface markers and thus would lose magnetic attachment. A single isolated stem cell is thus monitored through successive divisions.

The methods of the present invention can also be used for analyzing human and other mammalian cells. Single human or other mammalian cells can be isolated and retained in particular positions using surfaces patterned with molecules that bind cells. However, this involves dramatic remodeling of the cell surface to attach to the labeled substrate, and is probably only useful with “flat” or “stretchy” cells like epithelia. Circulating cells that are typically “rounder” and less likely to attach to a substrate can be readily isolated using the systems, components, devices and methods of the present invention. For example, magnetic beads are attached to cells and isolated using the magnetic capture sites of the present invention. Accordingly, attachment of cells to substrates is not required, thereby avoiding undesirable intracellular consequences arising from the effect of substrate attachment on inducing deleterious signal transduction pathways.

Essentially any cell that does not regularly turn over cell surface markers/proteins can be isolated using the systems, components, devices and methods of the present invention. In one embodiment it is desirable to analyze cells that are free to rotate in solution, for example to study cells that “swim” or are otherwise motile in a liquid phase without having to track their movement around a large area. Accordingly, one embodiment of the present invention includes magnetically labeling a motile cell, fluidically transporting the magnetically-labeled motile cell to a magnetic capture site of the present invention, applying a magnetic field within the magnetic capture site that is strong enough to maintain the motile cell within the magnetic capture site and weak enough to permit cell motion within the magnetic capture site. A suitable example of a motile cell is a flagellar cell. A flagellar cell is magnetically labeled as provided herein and fluidically transported to magnetic capture site. The magnetic field within the magnetic capture site is adjusted to permit motion of the flagellar cell within the magnetic capture site. A suitable optical system is capable of monitoring the motion of the flagellar cell swimming itself in circles as it is held within the magnetic capture site.

### EXAMPLES AND ILLUSTRATIVE EMBODIMENTS

Examples and illustrative embodiments of the systems, components, devices and methods of the present invention are provided herein.

### Example

Protocol for plating ferromagnetic material on PDMS. This method can be used on any layer of a multilayer device, allowing easy integration into microfluidic devices of the present invention.

Controllable magnetic trap. The magnetic trap (i.e., magnetic capture site) can be actuated using the methods described below to trap or release cells or molecules attached to paramagnetic or superparamagnetic particles (beads).

Overall system for automated lifetime or pedigree analysis of a cell. Provided herein is a system that can hold a single cell (mother) in place for lifetime monitoring while moving the division product (daughter) to a different location.

Optical detection of a cell division event. This ability allows automation of the pedigree analysis.

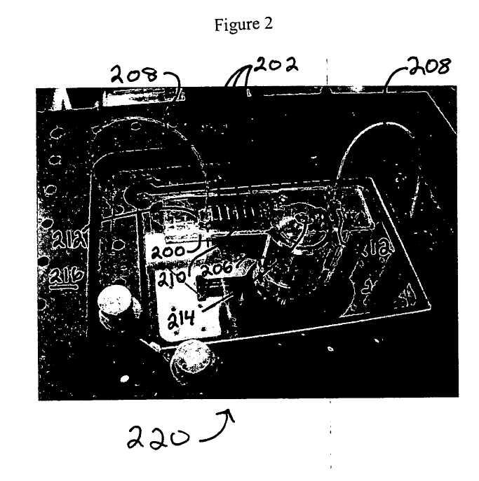

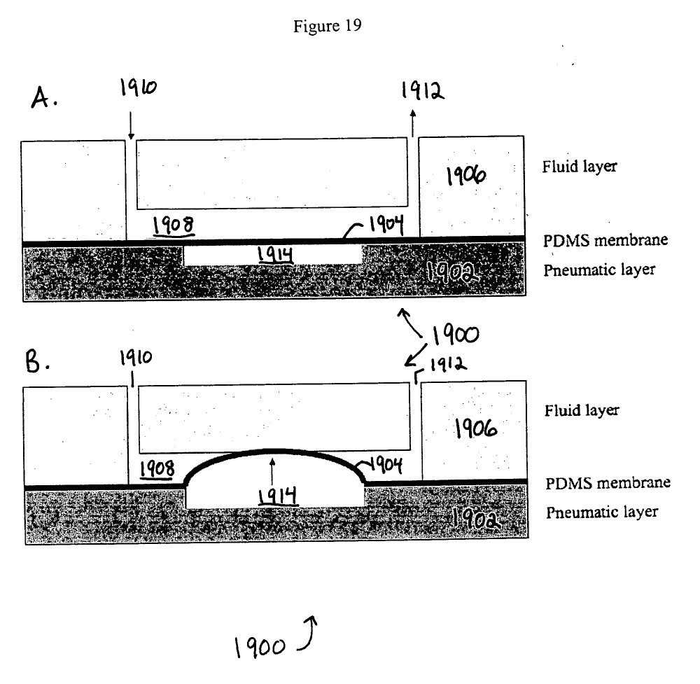

Microfluidic device. Referring to FIG. 2, a microfluidic device 200 is made of PDMS bonded to a 2″×3″ microscope slide. The microfluidic device resides on platen 204. The channels 210 are made using the protocol developed by Duffy et al. [9] and the valves (see FIG. 19) are made using the protocol in developed by Unger et al. [10]. A device incorporating multiple capture sites 202 and microfluidic valves (not shown) is fabricated according to these methods as described below. Tubing 208 fluidically transports fluid containing cells (not shown) to and from the microchannel 210 through inlet and outlet ports 212. Microscope objective 206 images a magnetic capture site 214 that is within the field of view of the microscope objective. FIG. 2 shows a microfluidic device 200 with multiple capture devices (magnetic capture sites 202) that was used as a microfluidic magnetic trapping device. The microfluidic device 200 may be disposable. It is set onto the device stage 204 above the optical system and objective 206. PEEK tubing 208 is inserted into each of the inlet and outlet ports 208. A single microfluidic device 200 is operated for the entire run of the experiment (i.e., from the time yeast culture is pumped into the device until the time of the last budding event at the longest-living mother cell.)

Multiple cells can be captured on a microfluidic device in two concurrent ways. First, the capture sites can be located in parallel using the valve scheme shown in FIG. 3. This allows each site to be addressed by an individual set of input valves 306 for controlling delivery of nutrients from a microchannel 302 and yeast cells from microchannel 304 to the mother cell capture site (magnetic capture site 308). Output valves 310 control the fluidic transport of waste to microchannel 312 delivery of a daughter cell to an agar plate (not shown) using microchannel 314. In this example, each site is monitored in a different field of view. The separation distance between the capture sites depends on the geometry of the channels, valves, and captures devices, but it could be as small as 2-5 mm. This allows 150-1000 capture sites to be placed on a single 2″×3″ glass slide. Using 6 capture devices per capture site allows capture of 900-6000 cells at the same time.

### Example

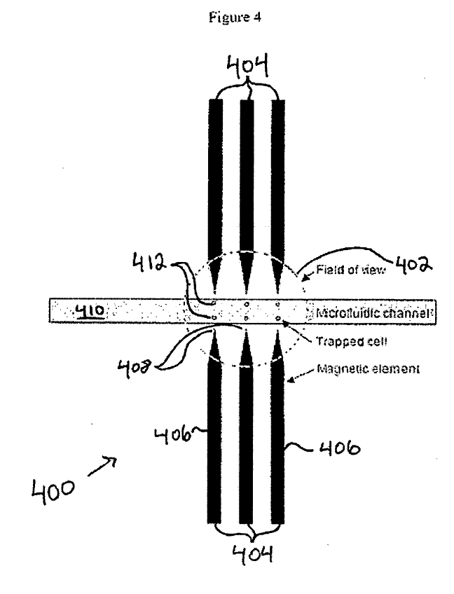

Referring to FIG. 4, one magnetic capture site 402 can trap multiple cells 412 in the vicinity of tips 408 of ferromagnetic structures 404. A magnetic field source (not shown) is applied external to the magnetic capture site 402 and external to the microchannel 410 near the portion 406 of the ferromagnetic structures 404. This allows multiple cells 412 to be trapped within the same field of view 402 (corresponding to the magnetic capture site). In this example, the magnetic capture site 402 is served by one set of valves (not shown). This arrangement is capable of detecting two or more cell budding events occurring at the same time. The capture devices are separated by 20 μm. FIG. 4 is not drawn to scale as a typical channel width can be about 100 μm and a typical yeast cell is about 5-10 μm in diameter.

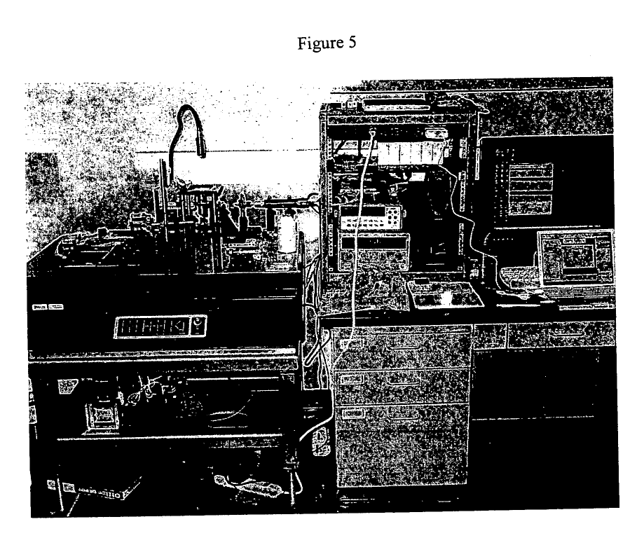

Optical system. Two examples of optical systems are described in detail. First is a fiber optic bundle system which is described in detail further below. A halogen lamp or an LED is used to illuminate the specimen. A microlens assembly or two back-to-back objectives image the specimen onto the tip of the imaging bundle. The specimen is focused by moving the z stage and translated by moving the xy stage (Newport Corp., Irvine, Calif.). The 10 ft long imaging bundle is a Sumitomo (Ofuna, Japan) IGN-08/30 silica image guide. The 10× objective and the stage attached to the camera tube focuses the image plane of the camera onto the tip of the fiber optic imaging bundle. This optical arrangement achieves a 40× or 80× total optical magnification at the CCD array of the camera. The camera (Watec America Corp., New York) provides a NTSC gray level signal to the digitizing card in the computer. The image capture card digitizes the 640×480 image with 8-bit resolution. Multiple sites can be monitored on the same device by observing each site with a microlens and a fiber optic bundle. Each fiber bundle is then observed with a camera and each camera is monitored by the software in parallel. The overall system is shown in FIG. 5. A view of the microfluidic device operating with the optical system and the fluid interconnects is shown in FIG. 2.

A second example of an optical system uses an automated motorized stage system. This uses a standard microscopy setup as depicted in FIG. 2. The microfluidic device 200 sits on a motorized stage 216 above a microscope objective 206 (10×, 20×, 40×, or 60×.) The objective 206 projects the image onto the plane of a CCD (not shown) as in (1). This system 220 is a higher resolution optical system than (1) because the image is not pixellated by a fiber optic bundle. This optical system typically monitors one or a few magnetic capture sites 214 simultaneously. Additional magnetic captures sites are monitored by scanning the stage 216 to move the microfluidic device 200 to scan each site.

Device construction. The following are the steps to construct the microfluidic device now being used:

1. Silanize a silicon or glass wafer for 30 minutes. This silanization step allows the PDMS to be peeled off of the substrate surface. The glass wafer can be unmodified or modified with photoresist channels. Photoresist channels allow the metal layer to be patterned on top of a channel layer for use in a multilayer device (for example, the control channel for a Fluidigm NanoFlex™ valve, www.fluidigm.com).

2. Spin PDMS (GE RTV 615A in 5:1 base:catalyst ratio) onto the wafer at 2600 RPM. Bake overnight to cure.

3. Pattern the ferromagnetic alloy on top of the PDMS (see section below).

4. Spin a PDMS (GE RTV 615A in 20:1 base:catalyst ratio) layer on top of the magnetic layer. Bake for 35 minutes at 80° C. to partially cure.

5. Cast a channel layer of PDMS (GE RTV 615A in 5:1 base:catalyst ratio) over a wafer coated with photoresist in the shape of channels. Bake for 75 minutes at 80° C. to partially cure.

6. Align the two layers under a microscope and press together. Bake overnight at 80° C. to fully bond.

7. Lift the metal layer off with the channel layer and bond to a glass slide or another layer of PDMS.

These steps are general knowledge and have been adapted from various microfluidics papers, including those from the Whitesides lab and the Quake lab. Ferromagnetic compositions are then electrolessly deposited. This allows the ferromagnetic compositions to be patterned into the desired ferromagnetic structures on the layer of choice.

Metal plating and patterning. The following is the general protocol for plating ferromagnetic compositions on PDMS. Film thickness is controlled by adjusting the length of time that the PDMS is covered in the plating bath (step 6).

1. Activate the surface of the PDMS by treating it in air plasma for 20 s.

2. Cover the surface in sensitizer (see below) for 5 minutes.

3. Rinse gently with deionized (DI) water.

4. Cover the surface in catalyst (see below) for 5 minutes.

5. Rinse gently with DI water.

6. Cover the surface in plating bath (see below) for 5 to 30 minutes.

Maintain the substrate at 38° C. during the plate.

7. Rinse in 38° C. DI water.

8. Rinse with ethanol.

9. Rinse with DI water.

10. Dry with nitrogen.

Sensitizer: 10 mg Tin(II) chloride, 1371 μl 0.1N HCl, 50 ml DI water

Catalyst: 10 mg Palladium (II) Chloride, 137 μl 0.1N HCl, 50 ml DI water

Plating bath: 50 ml DI water, 1.47 g sodium citrate tribasic dihydrate, 0.395 g Nickel(II) sulfate hexahydrate, 0.984 g Cobalt(II) sulfate heptahydrate, 7.5 mg thiodiglycolic acid, 0.071 dimethylamineborane. Heat to 48° C. and adjust pH to 7.00 with sodium hydroxide.

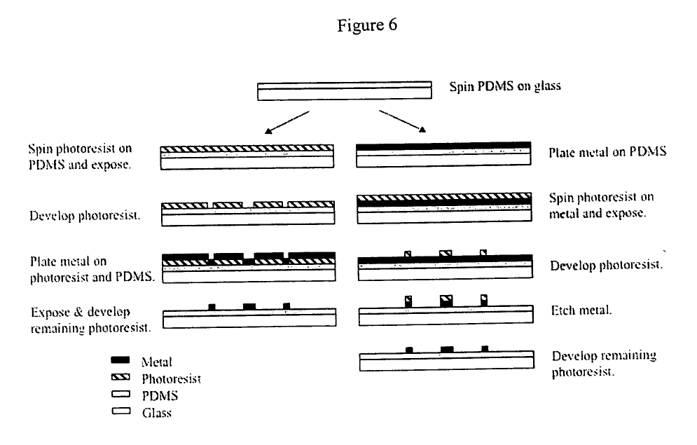

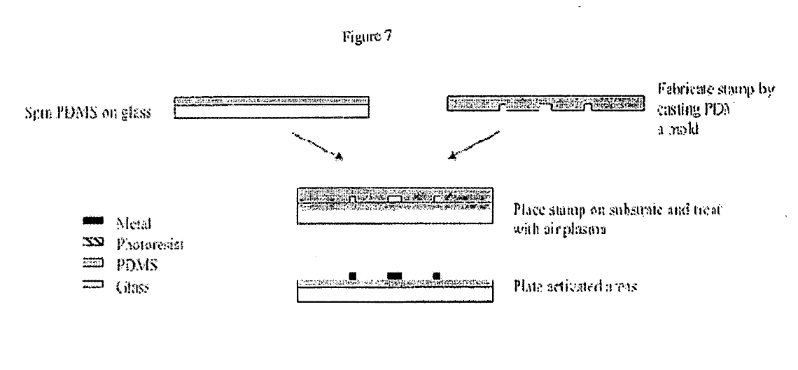

The following three methods have been tested to pattern Ni—Co—B alloy on PDMS. They listed in order from the highest resolution structures to the lowest resolution structures. A diagram describing the first two methods is shown in FIG. 6. A diagram describing the third method is shown in FIG. 7.

1. Photoresist as an etch mask.

a. Plate the entire surface of the PDMS as above.

b. Pattern photoresist SIPR-7120-5 onto the metal using standard photolithography procedures.

c. Etch the metal using a Ni etchant.

d. Develop off the remaining photoresist.

2. Photoresist as a deposition mask.

a. Pattern photoresist SIPR-7120-5 onto the PDMS using standard photolithography procedures.

b. Plate the entire surface of the wafer, coating both photoresist and exposed photoresist, as above.

c. Develop the photoresist to lift off the metal.

We have surprisingly discovered that a plasma can be advantageously used in a soft imprint lithography process of electrolessly depositing a ferromagnetic composition on a substrate. A surface (PDMS) is masked with a PDMS stamp or pattern. The masked surface is exposed to an air or oxygen plasma. The plasma treatment enhances deposition of a ferromagnetic composition. This process is particularly preferred as it is fast and requires less processing steps and processing chemicals than the more traditional photolithographic patterning.

3. Soft imprint lithography

a. Activate selected areas on the PDMS by placing a PDMS “stamp” on the surface and treating it in the air plasma. The stamp will block selected areas of the substrate; those areas typically are not activated by the plasma.

b. Plate as above. Only the areas exposed to the air plasma (i.e., not covered by the stamp) will be plated.

Any combination of the following elements could be deposited using this method: cobalt, iron, and nickel. This includes Permalloy, an iron-nickel alloy. To deposit other combinations of elements, substitute the correct ratio of iron sulfate, cobalt sulfate, or nickel sulfate for the cobalt sulfate and nickel sulfate of the plating bath. All elements are in their sulfated versions for the reduction by dimethylamineborane to occur. Boron will be deposited with each alloy as a result of the reaction. The cobalt-nickel combination has been tested.

The metal plating and patterning has been tested on the following PDMS formulations: Dow Sylgard 184 and GE RTV 615 in 5:1, 10:1, and 20:1 base to catalyst ratios. This process can also be used to deposit ferromagnetic compositions on other substrates besides PDMS.

Controllable magnetic trap: The yeast (attached to paramagnetic beads) is trapped using magnetic force created by the sharp gradient generated by an externally magnetized tip. The tip fabrication is described above. The tips now being tested are 20-40 μm wide, 5 mm long, and an estimated 100 nm thick. The tip can be actuated (magnetized) by any of the following methods:

A permanent magnet is rotated 90 degrees. When the magnet is aligned with the tip, the field and the gradient formed are strong enough to capture the cell. When the magnet is aligned perpendicular to the tip, the field and the gradient are not strong enough to capture the cell and the cell is released. This method of actuation has been tested successfully. The magnet could be attached to the axis of a simple motor or solenoid so that the computer could control the actuation.

A miniature electromagnet is aligned with the tip. When current flows through the electromagnet, the tip is magnetized and the yeast cell is trapped. When current stops, the tip is not magnetized and the yeast cell is released.

Wires are fabricated on layers of PDMS surrounding the tip to form an electromagnet embedded within the PDMS. The electromagnet's core is the tip itself. The wires can be fabricated on PDMS using gold deposition methods. The electromagnet operation is described above.

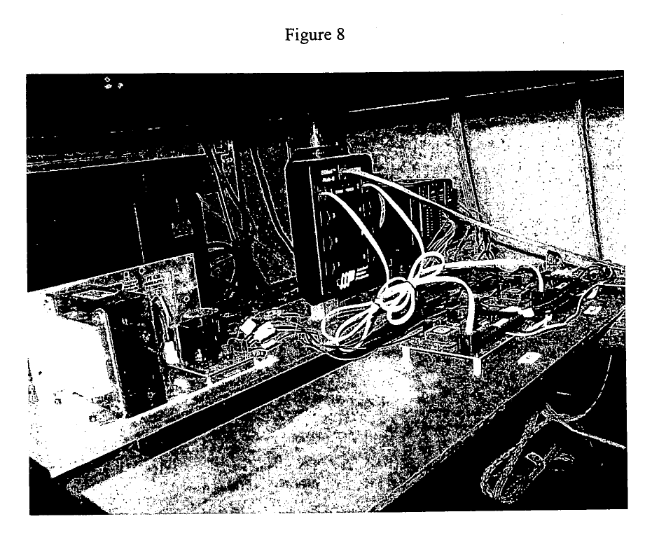

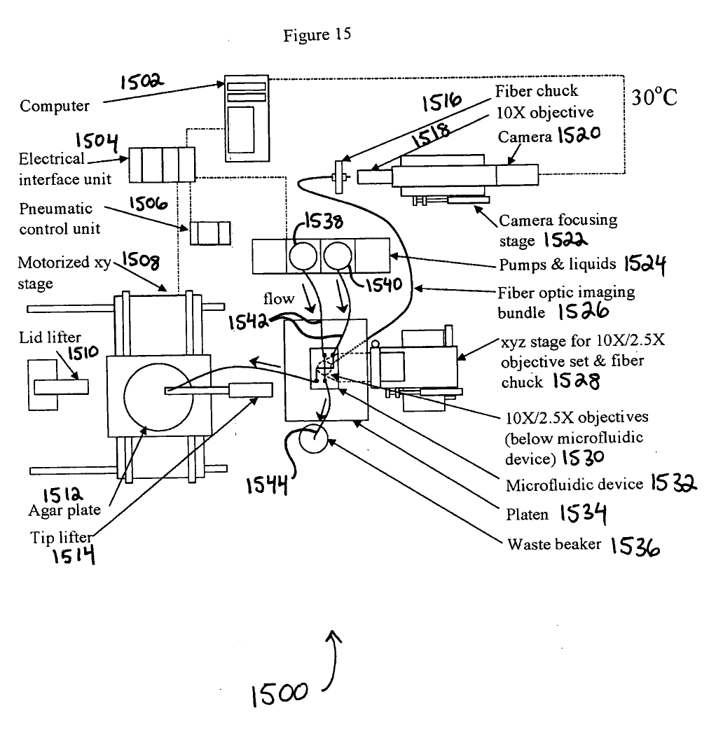

Auxiliary systems: Referring to FIG. 15, a tip lifter 1514 and lid lifter 1510 provided from commercial products can be motorized or pneumatically actuated. The motorized stage 1508 is also a modified commercial product (salvage unit, manufacturer unknown) and is driven by commercially available motor drivers (Applied Motion Products, Inc., Watsonville, Calif.). The electrical interface unit 1504 is a commercially available Ethernet hub and analog and digital input and output cards (Optimation, Huntsville, Ala.) (see photograph in FIG. 8 of this electrical equipment). The pumps 1524 (Global FIA, Fox Island, Wash.) are commercially available.

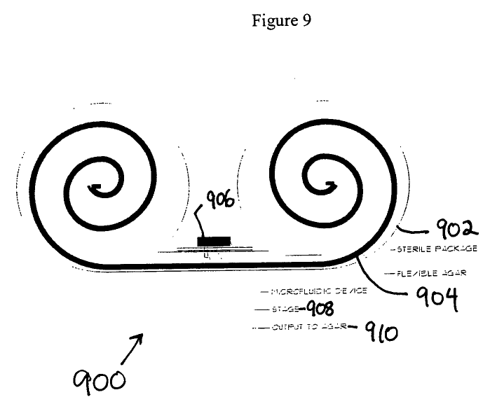

Referring to FIG. 9, a moving band of agar 904 is mounted onto the microfluidic device stage 908, which is an alternative to the tip lifter/lid lifter/motorized stage configuration depicted in FIG. 15. The moving agar band 904 eliminates much of the equipment and shortens the distance the daughter cell (not shown) needs to travel from the microfluidic device 906 to the agar. FIG. 9 shows one example of this configuration, the “agar reel” 904. The reel is placed under the device 906 and the agar is exposed to the outlet port 910 of the device 906 when the daughter buds. After the daughter is placed on the agar, the reel “winds” to the next position.

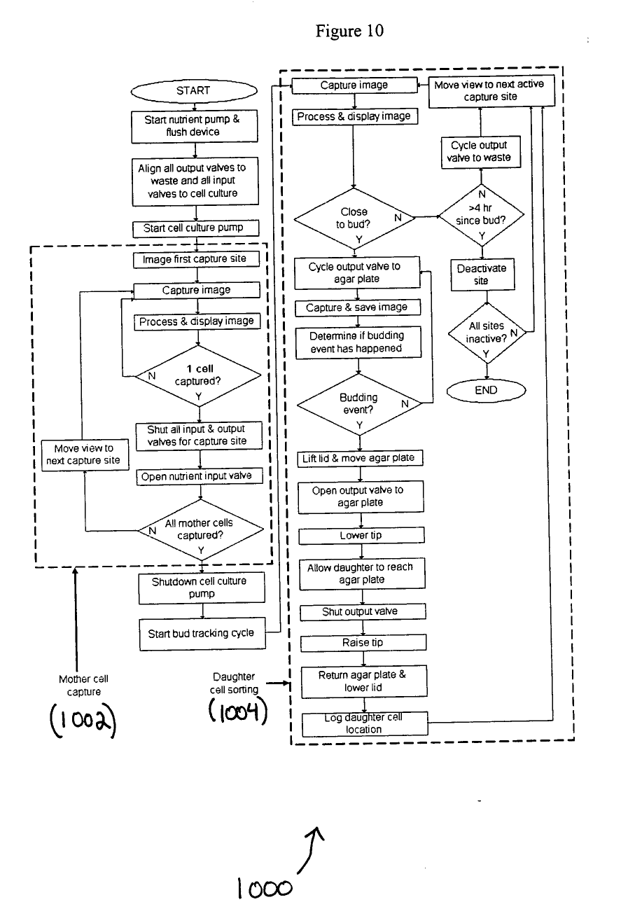

Software: The software was written in C++. FIG. 10 provides the software flow chart 1000. In the following description of operation, also refer to FIG. 3. This software accounts for multiple capture sites, and multiple ferromagnetic structures at each site. Before the operation begins, the yeast cells are biotinylated and attached to magnetic beads. Alternatively, the cells can be mixed with biotin and then mixed with beads on the microfluidic device upstream of the capture sites.

To start the operation, start the nutrient pump and flush all air out of the device and prime all channels with liquid. Align the output valves for every capture site to waste and the input valves for every capture site to cell culture. Actuate every capture device. Start the cell culture pump. Cell culture will now be flowing by every capture device to waste. Subroutine 1002 captures the mother cell(s). View the image of the first capture site. Process the image to determine if capture has taken place. If greater than one cell is captured at a single capture device, release and re-actuate the capture device. If one cell is captured at each capture device, shut all output and input valves for the site, and open the nutrient input valve. Move the view to the next capture site. When all capture sites have captured cells, shut down the cell culture pump and start the bud tracking cycle.

Subroutine 1004 sorts the daughter cells. View the first capture site. Process the image and determine if the cell is close to budding. If a budding event will occur soon, open and shut the output valve to the agar plate and determine if the budding event has occurred by processing the image and determining if the daughter cell has separated from the mother cell. If the budding event has occurred, lift the agar plate lid, move the agar plate into position, open the output valve to the agar plate, lower the tip, and allow the daughter cell to reach the agar plate. Then shut the output valve, raise the tip, and return the agar plate to its standby position, and lower the lid. Log the daughter cell location and move the view to the next active capture site. When it has been more than 4 hours since the most recent budding event at a capture site, the site is labeled inactive. When all sites are labeled inactive, the operation is complete.

System applications: The yeast pedigree analysis system is not limited for application using yeast. It can be used in any application where magnetic beads can be attached to a cell wall, microorganism, particle or to another molecule of interest. For example:

Pedigree Analysis: Pedigree and lifetime analysis of a single cell that generates a new cell wall during division. This allows the old cell wall to retain its beads and remain trapped.

DNA sequencing. A single strand of DNA is attached to a magnetic bead and held in place while a “sequencing by synthesis” analysis (e.g., pyrosequencing or 4-color DNA sequencing) occurs. During these procedures, mixtures containing nucleotides delivered by microfluidic channels to the single strand of DNA.

DNA synthesis. The strand undergoing synthesis is attached to a magnetic bead and held in place during elongation. The microfluidic channel is used to deliver reagents to the strand.

Electroporation. The cell is held in place, and a voltage is applied to the magnetic element. The pores of the cell open, and vectors can be delivered into the cell via the microfluidic channel.

Device testing: The microfluidic device has successfully captured a single budding and non-budding yeast cell. Long-term testing of the device is ongoing. The optical system has been successfully tested. Further details of the optical and image analysis systems are provided below.

Fiber Optic Imaging Bundle and Image Analysis System: An imaging system has been designed and built for yeast pedigree analysis. The system uses a fiber-optic imaging bundle to recognize single yeast cells. Image processing software has been developed to accurately classify the cells as either budding or not budding a daughter cell. This system is intended to replace the body of a microscope for the detection of budding in a microfluidic system.

This imaging system is integrated into a microfluidic system that automates yeast pedigree analysis. The microfluidic device can hold a yeast cell in place and deliver each bud to a specified location on an agar plate. Image analysis detects that the mother cell has been trapped, and then detects two times per cycle (˜90 minutes per cycle) to detect that the mother cell is budding and to detect the budding event. Pedigree analysis is performed on 10-100 mother yeast cells in parallel. The microfluidic device is 2″ by 3″, leaving minimal space for conventional microscope optics to monitor every holding chamber on the device. Fiber optic imaging bundles provide a dense packing alternative for continuous observation of all budding events in parallel.

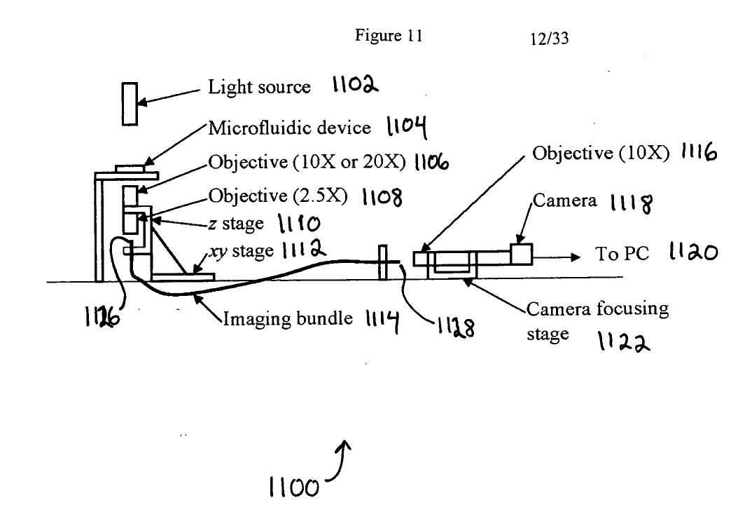

A schematic of the single channel test hardware setup is shown in FIG. 11. A halogen lamp 1102 is used to illuminate the specimen (not shown). Two objectives 1106, 1108 are placed back-to-back to image the specimen onto the tip 1126 of the imaging bundle 1114. The objective 1106 nearest the specimen is either 10× or 20× (both have been tested) and the other objective 1108 is 2.5× for a total of 4× or 8× magnification at the tip of the fiber. A microlens can replace these two objectives. The specimen is focused by moving the z stage 1110 and translated by moving the xy stage 1112. The 10 ft long imaging bundle 1114 is a Sumitomo (Ofuna, Japan) IGN-08/30 silica image guide. The 10× objective 1116 and the stage 1122 attached to the camera tube 1118 focuses the image plane of the camera onto the tip 1126 of the fiber 1114 optic imaging bundle. This optical arrangement achieves a 40× or 80× total optical magnification at the CCD array (not shown) of the camera. The camera provides a NTSC gray level signal to the digitizing card in the computer 1120. The image capture card (not shown) digitizes the 640×480 image with 8-bit resolution. The image processing software was written in the C programming language. The yeast strain was obtained from the Fred Hutchinson Cancer Research Center (FHCRC—http://www.fhcrc.org/) in Seattle, Wash. Cells were grown and stored on growth media at 4° C., then put into liquid nutrient at 30° C. for 12 hours. The tested samples were diluted in water to a level where 1 to 10 cells appeared in the image frame when placed on glass slides.

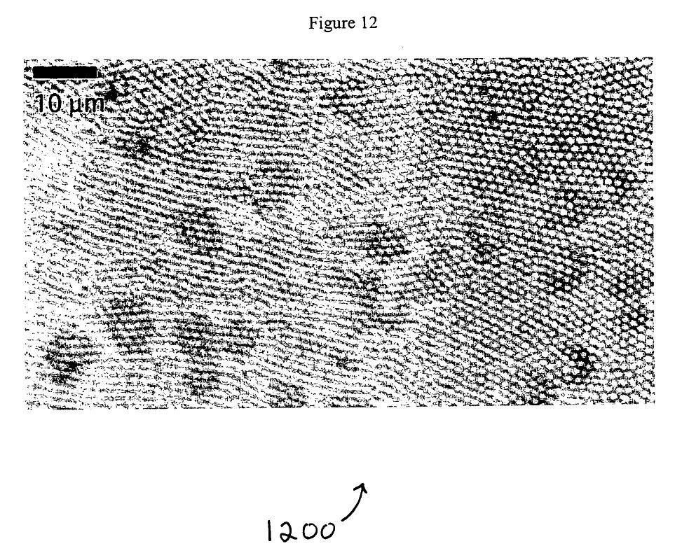

The imaging bundle used in the experiment contains 30,000 fiber cores located 2.21 μm on center. The unprocessed image in FIG. 12 is of yeast taken with a lower resolution imaging bundle that contains fiber cores located 8 μm on center. This image illustrates the two major problems most commonly encountered when using a fiber-optic imaging bundle. First, each of the core fibers is effectively a pixel transmitting only one intensity level. This results in a coarse discretization of the image. Second, the cladding around each of the core fibers results in a honeycomb effect that spans the entire image. This cladding is at approximately the same gray level as the perimeter of the yeast cells. The discretization and the honeycomb make many standard image processing algorithms, including edge detection, automatic thresholding, and background detection, very difficult or unusable. To overcome this, an image processing algorithm was developed and implemented, as follows:

Step 1: A blank slide or empty microfluidic device is placed on the specimen stage. A background image is captured and segmented into an 8×8 grid (80×60 pixels), then the threshold value, the mean minus one half the standard deviation, is calculated for each of the 64 sections. The background is segmented to account for uneven illumination over the frame and slight changes in illumination over the 60 hour lifetime of the pedigree analysis.

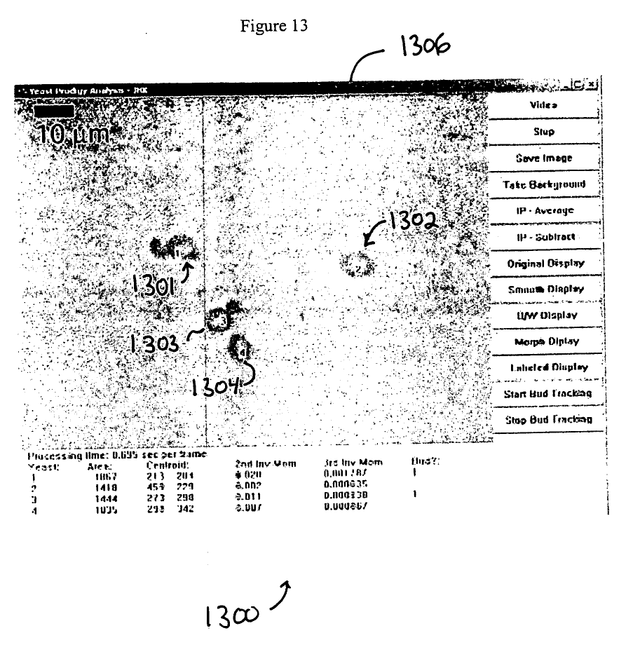

Step 2: The slide or microfluidic device containing yeast is then placed on the specimen stage. The user focuses the specimen, then starts the real-time processing. The user can select to display the captured image or the processed image (not shown) at any stage of the algorithm by clicking the appropriate display button (see display 1306 in FIG. 13). FIG. 13 is a display screen of an embodiment of an image analysis system used in analyzing yeast images with user interface as seen through an imaging bundle in 1300 with fiber cores located 2.21 mm on center. Two budding (#1 1301 and #3 1303) and two non-budding yeast cells (#2 1302 and #4 1304) are accurately identified. The scale bar refers to the specimen plane.

Step 3: The image is filtered with a 7×7 boxcar filter. This smoothes the honeycomb pattern and raises the gray level of most of the dark cladding pixels to above the level of the local threshold. The image of the yeast cells loses some resolution, but the resolution is still adequate to recognize a bud.

Step 4: The smoothed image is then changed into a binary image (white cells and black background) using the threshold value at each of the 64 sections of the 8×8 grid. Some of the honeycomb still remains as groups of white pixels, but the yeast cells are mostly intact

Step 5: White areas are eroded by moving the center of a 5-pixel radius disc along the boundary of each white area and blackening pixels that overlap with the disc. Any regions smaller than the disc (most of the non-yeast cell noise) are removed. The remaining white areas are then dilated by moving the center of the disc along the boundary of the eroded white regions and whitening pixels that overlap with the disc. This dilation restores the size and general shape of the yeast cells.

Step 6: The operations in step 5 are reversed. The white areas are first dilated using the 5-pixel radius disc to bridge small gaps in the yeast cells. To restore the size and general shape of the yeast cells, the dilated areas are then eroded using the same disc.

Step 7: The image is segmented into regions using a modification of the 4-adjacency sequential connected pixel algorithm. (Azriel Rosenfeld and John L. Pfaltz, Journal of the Association for Computing Machinery 13, 471 (1966)). The area of each region is calculated and those regions that are greater than 400 pixels, equivalent to a 2 μm diameter yeast cell, are kept in the image.

Step 8: Each yeast cell is classified as budding or non-budding. A bud on a yeast cell will cause a lopsided peanut shape, which will be reflected in the 2nd and 3rd moments:

$${μ}_{pq} = {\sum }_{x} {\sum }_{y} {( x - \overline{x} )}^{p} {( y - \overline{y} )}^{q} u ( x , y ) \qquad ( 1 )$$

where ( x̅, y̅) is the centroid of the yeast cell, u(x,y) equals one for each pixel of the yeast cell and equals zero otherwise, and p=1, 2, 3, and q=1, 2, 3. Hatamian's algorithm (Mehdi Hatamian, IEEE Transactions on Acoustics, Speech, and Signal Processing 34, 546 (1986)) is used to find the central moments of each yeast cell. These central moments are translation-invariant only. The more general size and rotation-invariant moments, necessary for a growing and rotating yeast cell, are found by using Hu's method (Ming-Kuei Hu, IRE Transactions on Information Theory IT-8, 179 (1962)). Each central moment, μ_{pg}, is normalized to its size-invariant moment, η_{pq}, by dividing the central moment by the appropriate power of the area, μ₀₀:

$${η}_{pq} = \frac{{μ}_{pq}}{{μ}_{00}^{γ}} , \\ γ = \frac{p + q}{2} + 1 \qquad ( 2 )$$

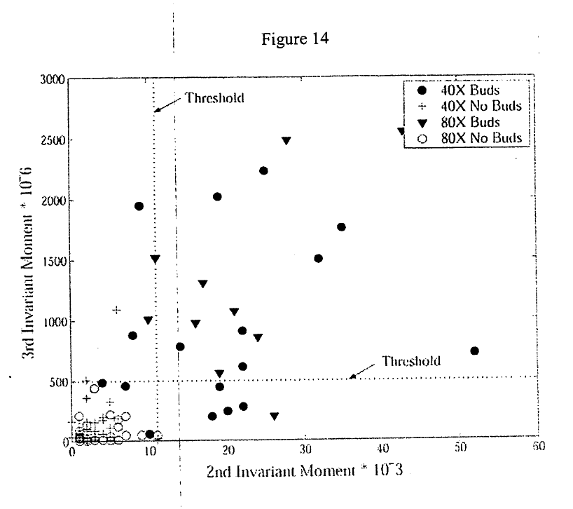

Hu derives six invariant moments. The 2^{nd} and 3^{rd} invariant moments, X and Y, were chosen for classification because they were found to vary significantly among yeast cells.
X=(η₂₀−η₀₂)+²4η₁₁² (3)
Y=(η₃₀−3η₁₂)²+(3η₂₁−η₀₃)² (4)
To determine the threshold levels to classify a yeast cell as budding or non-budding, the 2^{nd} and 3rd invariant moments were individually calculated for 58 cells under 40× total magnification and 34 cells under 80× total magnification. The cells were a random mix of budding and non-budding cells. From this data, the following threshold was set: if the 2^{nd} invariant moment is greater than 11×10⁻³ OR if the 3^{rd} invariant moment is greater than 500×10⁻⁶, then the yeast cell is classified as a budding yeast cell (FIG. 14). Counting occurrences beneath the thresholds predicts the following performance: 0% false negatives with 4% false positives under 80× magnification, and 12% false negatives with 7% false positives under 40× magnification.

The system was tested for its ability to recognize a yeast cell and to classify the yeast cell as budding or non-budding. Using the 80× total magnification setup, a glass slide containing yeast was randomly scanned at different locations using the xy stage. Each picture was chosen to contain at least 2 yeast cells and a mix of budding and non-budding yeast cells. The same experiment was repeated using the 40× total magnification without altering the algorithm. Three total slides were used with slightly different lighting conditions. The system was used to successfully recognize all 258 yeast cells with no false positives. Tables 1 and 2 show the better than predicted results for the bud classification. The four cases where two or more unique cells overlapped were removed from the reported results and counted as outliers; this condition was incorrectly classified by the system as budding yeast. Overlapping cells will not occur in a controlled yeast pedigree analysis microsystem. Frame processing time was acceptable: less than 0.5 seconds for 1 or 2 yeast cells and approximately 0.9 seconds for 8 yeast cells.

TABLE I
80X Test results. All yeast cells were correctly classified as budding
or not budding a daughter cell.

Ground Truth

| Test Result | Software Predicted | Bud | No Bud |
|---|---|---|---|
| Bud | 25 | 25 | 0 |
| No Bud | 47 | 0 | 47 |

TABLE II
40X Test results. 1 of 41 budding yeast cells was incorrectly
classified (2% false negatives). 5 of 115 non-budding yeast
cells were incorrectly classified (4% false positives); these 5
yeast cells were located near a flaw in the imaging bundle that
was segmented as part of the yeast cell.

Ground Truth

| Test Result | Software Predicted | Bud | No Bud |
|---|---|---|---|
| Bud | 45 | 40 | 5 |
| No Bud | 111 | 1 | 110 |

A successful imaging system has been designed and built for yeast pedigree analysis. Other uses include applications where cell division is monitored at many locations on the same agar plate, glass slide, or microfluidic device, and multiple imaging bundles replace a microscope combined with a motorized xy stage. For example, since yeast budding starts at the same time DNA replication starts, researchers could automatically monitor the cell cycle stage distribution at multiple points on the same agar plate.

### Example

System Architecture. FIG. 15 shows the overall system architecture of this example. The system can be divided into five sub-systems: the imaging system, which comprises the optical train and the image processing software; the input station, which comprises the liquid inputs and the pumps; the microfluidic device; the output station, which comprises the agar plate, the lid lifter, the motorized xy stage, the waste beaker, and the tip lifter; and the pneumatic system. The system is controlled by automation software written from the ground up in C++. The system is bolted to a vibration isolation table. The system can be enclosed in an acrylic box and heated to 30° C. for yeast growth.

Operational Protocol. Before the system is started, the yeast culture is prepared. A yeast colony on a YEPD (a yeast nutrient) agar plate is grown for approximately 15 hours in liquid YEPD at 30° C. When the yeast concentration reaches 10⁷ cells/ml, the yeast is biotinylated and attached to streptavidin coated paramagnetic beads. Streptavidin is a tetrameric protein that binds very tightly to biotin, a small molecule. The loose paramagnetic beads are then separated out and the culture is diluted to 10⁵-10⁶ cells/ml. Cell concentration is determined using a glass slide hemacytometer.

The yeast culture bottle is then attached to the pump inlet. All remaining actions are automated. The culture is first pumped into the microfluidic device. The microfluidic valves are initially aligned to output to the waste beaker. A single cell, the mother yeast cell, is captured and held at the cell trapping point directly over the fiber optic imaging bundle and magnifying objectives. After the image processing software has detected the trapping of the cell, the valves controlling the input switch from yeast culture to yeast nutrient. The valves controlling the output from the microfluidic system continue to direct the nutrient flowing past the trapped mother cell to waste.

The mother yeast cell will bud every 90 minutes while it is trapped. When the image processing software has detected a budding event, the output valves are aligned temporarily to direct the nutrient past the mother cell to the agar plate. The daughter cell flows with the nutrient, through the output channel, through the output PEEK tubing held by the tip lifter, and to the designated location on the agar plate. The output valves are then realigned to waste and the process repeats for the next detected bud.

The agar plate is aligned to the correct location using an xy-stage that is moved by two stepper motors. In between buds, the agar plate is capped to maintain hydration of the media. When the budding event is about to occur, the lid lifter, a vacuum suction unit that moves in the z-direction, lifts the lid. The agar plate moves into position. The tip lifter, which moves in the z-direction only, drops the tip of the PEEK tubing to within 1 mm of the agar plate. Surface tension pulls the drop containing the daughter yeast cell onto the agar plate. The tip is then lifted and the agar plate moves back into place and is capped in preparation for the next budding event.

Imaging system. Fiber-optic imaging bundles are a class of fiber optics that maintain coherence of the image by using thousands of individual optical fibers, each fiber analogous to a pixel on a television screen. Imaging bundles were used in this system to eliminate the massive body of a traditional microscope and allow multiple points on a device to be monitored in parallel.

FIG. 11 shows the imaging system optical train. A halogen lamp 1102 is used to illuminate the microfluidic device; this can be replaced by a single LED. Two objectives 1106, 1108, placed back-to-back, image the specimen onto the tip 1126 of the imaging bundle 1114. The objective 1106 nearest the microfluidic device 1104 is either 10× or 20× and the other objective 1108 is 2.5× for a total of 4× or 8× magnification at the tip of the fiber. A microlens optical train can replace these two objectives. The magnetic capture site (not shown) of the microfluidic device 1104 is focused by moving the z stage 1110 and translated by moving the xy stage 1112. All optical train stages were manual but can be upgraded to fully enable automatic control for autofocus and targeting features. The 3 m long imaging bundle 1114 is a Sumitomo (Ofuna, Japan) IGN-08/30 silica image guide. The optical train is completed by the addition of a 10× objective 1116 at the CCD camera end 1128 of the imaging fiber bundle 1114. A stage 1122 attached to the camera tube 1118 is used to focus the image plane of the camera onto the tip of the fiber optic imaging bundle. This optical arrangement achieves a 40× or 80× total optical magnification at the CCD array of the camera. The ⅓″ CCD camera (Watec, Yamagata, Japan) provides a NTSC gray level signal to the frame grabber card (not shown, CyberOptics, Beaverton, Oreg.), which digitizes the 640×480 image with 8-bit resolution.

The imaging bundle used in the experiment contains 30,000 fiber cores located 2.21 μm on center. The unprocessed image in FIG. 12 is of yeast cells taken with a lower resolution imaging bundle that contains fiber cores located 8 μm on center. This image illustrates the two major problems most commonly encountered when using a fiber-optic imaging bundle. First, each of the core fibers is effectively a pixel transmitting only one intensity level. This results in a coarse discretization of the image. Second, the cladding around each of the core fibers results in a honeycomb effect that spans the entire image. This cladding has approximately the same gray level as the perimeter of the yeast cells. The discretization and the honeycomb make many standard image processing algorithms, including edge detection, automatic thresholding, and background detection, very difficult or unusable. To overcome this and fully demonstrate robust event detection, a six-step image processing algorithm was developed and implemented in this research. The image analysis used is described above.

Input Station. The input station comprises an Instech (Plymouth Meeting, Pa.) P625 Peristaltic Pump and a bottle for each of the two inputs: yeast culture and nutrient (YEPD). Fluidic connections are achieved using 1/32″ OD PEEK tubing and Upchurch Scientific (Oak Harbor, Wash.) fittings. Each pump is individually controlled by software. A single syringe pump will be used to precisely place daughter cells on the agar plate.

Microfluidic Device. The microfluidic device is fabricated using a method introduced by Quin, et al. (“Rapid prototyping of complex structures with feature sizes larger than 20 um,” Advanced Materials, vol. 8, pp. 917-&, 1996). A mask of the device is drawn on AutoCAD (Autodesk, San Rafael, Calif.) and printed on thick transparency paper using a 20,000 dpi printer. 50 μm channels are then fabricated on a silicon wafer out of SU-8 (MicroChem, Newton, Mass.) using standard ultraviolet photolithography. The wafer is then treated with (tridecafluoro-1,1,2,2-tetrahydrooctyl)-1-trichloro silane in a vacuum chamber for 30 minutes. Optically clear silicone elastomer, polydimethyl siloxane (PDMS) (Dow Corning Sylgard 184), is poured over the SU-8 mold to form the channels. The cast PDMS is irreversibly bonded to glass or another layer of PDMS by treating both layers in a plasma cleaner (Harrick, Ossining, N.Y.) for 45 seconds, then pressing the two layers together.

The microfluidic device used in this system comprises three layers of PDMS: the top layer contains the fluid channels, the middle layer is a thin membrane that forms the base of the fluid channels and the top of the pneumatic channels, and the third layer contains the pneumatic channels that are used to open and shut the valves. This valve structure is modeled after the structure introduced by Hosokawa and Maeda (“Low-cost technology for high-density microvalve arrays using polydimethylsiloxane (PDMS),” presented at Micro Electro Mechanical Systems, 2001. MEMS 2001. The 14th IEEE International Conference on, 2001). A vacuum in the pneumatic channel pulls the membrane into the pneumatic layer and away from an obstacle in the fluid channel layer, opening the valve. Returning the pneumatic channel to atmospheric pressure closes the valve.

The yeast cell capture methods exploit the strong bond formed between streptavidin and biotin. The yeast cells are incubated with biotin (Pierce, Rockford, Ill.) before they are introduced into the microfluidic device. This biotin will remain attached to the mother yeast cell wall during yeast pedigree analysis, but the daughter cell that buds off the mother cell will be biotin-free. Therefore, a streptavidin-coated object will select for the mother cell.

One method of capture is to attach the biotinylated yeast cells to streptavidin-coated paramagnetic beads before pumping the yeast into the microfluidic device. The cells can then be magnetically captured. In the other cell capture method, the channel is selectively coated with streptavidin. The biotinylated cell then sticks to the surface of the channel.

Output Station and Pneumatic Control. The output station consists of a motorized xy stage, and two pneumatically controlled devices: a tip lifter and a lid lifter. The pneumatic control system consists of a manifold of solenoid actuated valves connected to the building vacuum system. Vacuum tubing connects the solenoid valves to the vacuum ports on the microfluidic device, and the tip lifter and lid lifter.

Automation Software and Interface. Software for automation control and image processing was written in C++ in the Microsoft (Redmond, Wash.) Visual C++ 6.0 developing environment. The software runs on a personal computer with a Microsoft XP operating system. Analog and digital inputs and outputs, including pump speed and valve position, are transmitted to and from the computer via Ethernet using an Optimation (Huntsville, Ala.) Optilogic remote terminal unit and bases.

Results: Yeast Detection and Classification. The imaging system was tested for its ability to recognize a yeast cell and to classify the yeast cell as budding or non-budding. Using the 80× total magnification setup, a glass slide containing yeast was randomly scanned at different locations using the xy stage. Each picture was chosen to contain at least two yeast cells and a mix of budding and non-budding yeast cells. The same experiment was repeated using the 40× total magnification without altering the algorithm. Three total slides were used with slightly different lighting conditions. The system was used to successfully recognize all 258 yeast cells with no false positives. Tables 1 and 2 above show the results for the bud classification.

The system accurately classified all yeast cells under 80× total magnification. In the 40× total magnification case, all but 1 of 41 buds were identified, but 5 cells that were not budding were classified as budding. All 5 of these yeast cells were located near a flaw in the imaging bundle that was segmented as part of the yeast cell. The four cases where two or more unique cells overlapped were removed from the reported results and counted as outliers; this condition was incorrectly classified by the system as budding yeast. Overlapping cells typically do not occur in a controlled yeast pedigree analysis microsystem.

The data collection and analysis time constraints were quite modest for this measurement. Frame processing time is less than 0.5 seconds for 1 or 2 yeast cells and approximately 0.9 seconds for 8 yeast cells. This processing time is acceptable for the yeast pedigree analysis system, which will monitor the status of 1 or 2 yeast cells at a time.

The fiber-optic imaging bundle provides the useable magnified size of the yeast cell compared as the diameter of each fiber core. Greater magnification onto the fiber tip results in a higher resolution image of the yeast cell, but greater magnification also narrows the field of view. The diameter of a normal yeast cell is between 4 and 10 μm and the diameter of the Sumitomo IGN-08/30 core is 2.21 μm. Under 4× magnification at the tip of the imaging bundle near the specimen, there are between 7 and 18 fiber cores across the width of each yeast cell. Lesser magnifications segment the cell so coarsely that the image analysis algorithm does not readily distinguish the yeast cell from fiber cladding. The magnification is typically not raised far above 4× because the field of view.

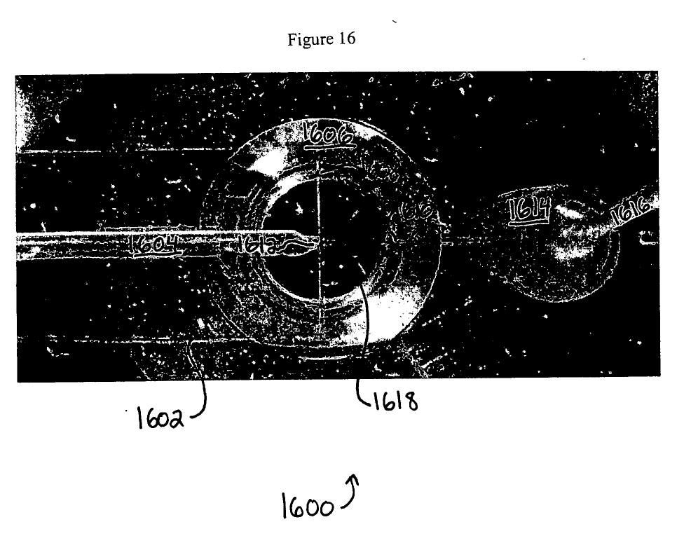

FIG. 16 is a photograph of an embodiment of a portion of a microfluidic device 1600 of the present invention. The permalloy wire 1604 (left) is embedded in the PDMS 1602. The channel inlet 1614 (right, attached to PEEK tubing 1616) is fluidically connected to microchannel 1610, which splits in a T-junction 1618 at the permalloy tip 1612. A 10× objective 1606 sits under the microfluidic device 1600. The microchannel width is 200 μm.

Yeast Cell Capture. Cell capture methods that have been used include attaching streptavidin-coated paramagnetic beads to biotinylated yeast. To capture the yeast cell, a sharp magnetic gradient is formed in the lumen of a microchannel. The force generated on a single paramagnetic bead is proportional to the gradient of the square of the magnetic field intensity.

Several methods of forming this gradient have been tested; two of these are preferred. In the first, a sharpened piece of permalloy wire is embedded in the microfluidic device. FIG. 16 shows a microfluidic device used to test this method. This wire is then magnetized using an external rare earth magnet. In the second method, an electromagnet made of a sharpened piece of permalloy wound with copper wire is embedded in the microfluidic device. In both methods, ridges formed of SU-8 are used to align the tip of the permalloy wire less than 200 μm away from the end of the channel during PDMS casting.

Modeling using MATLAB (The Mathworks, Natick, Mass.) showed that the force generated by the magnets using both methods is strong enough to hold the yeast cell in place, but the force drops off quickly in the first 100 μm from the tip. The major barrier to cell capture has been the difficulty in accurately positioning the tip of the permalloy wire less than 100 μm from the microfluidic channel during fabrication while still maintaining the integrity of the channels.

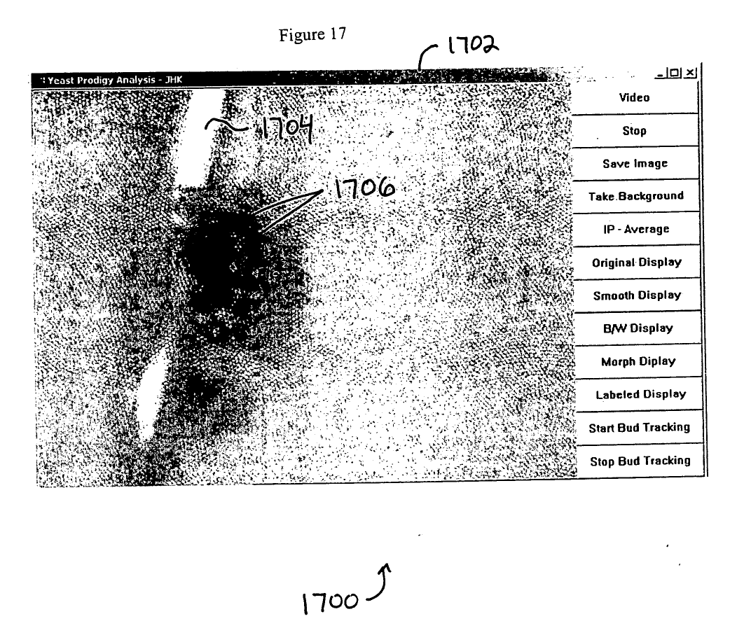

The prototypes that have been built can routinely capture paramagnetic beads. FIG. 17 shows a display 1702 of an image analysis system 1700 that shows the capture of 4.5 μm paramagnetic beads 1706 (Spherotech, Libertyville, Ill.) in a microfluidic device using externally magnetized permalloy wire. The microchannel wall of the microfluidic device appears as the white line 1704 running vertically through the frame. A Permalloy wire tip (not shown) was approximately 200 μm to the left of the channel wall. 0.8 μm paramagnetic beads (Seradyn, Indianapolis, Ind.) have also been captured using this method.

The yeast detection and classification system and the microfluidic system are operational. Methods of cell trapping have been tested. Single channel performance has been demonstrated. The system can be expanded to perform pedigree analysis on multiple yeast cells in parallel. The fluidic and optical elements used in the completed system have applications beyond that of yeast pedigree analysis as described above. Additional analyses, such as oxygen diffusion measurements and green fluorescent protein microscopy, can also be incorporated in these systems.

### Example

Overall system architecture are provided in FIG. 15 and described above. Automation software flow diagram is provided in FIG. 10 and described above. Imaging system and image analysis methods are also described above.

Microfluidic Device. Polydimethyl siloxane (PDMS, Dow Corning, Midland, Mich.) was chosen as the material for the microfluidic device because it is optically transparent, it can easily be bonded to itself, and its elasticity allows its use as a valve membrane material. In addition, the surface can be changed from hydrophobic to hydrophilic by treating it for 15 s or longer in the plasma cleaner (Harrick, Ossining, N.Y.). PDMS microfluidic devices were fabricated using a rapid prototyping method as described above. First, a mask is drawn using a computer aided drafting (CAD) program. Then, the mask is printed on a high-resolution printer. The photoresist is spun on a silicon wafer and exposed to UV light through the mask. The unexposed (negative) or exposed (positive) photoresist is then washed away using the photoresist developer. Finally, PDMS is poured and cured on the remaining structure to create microfluidic channels. The layer of PDMS can be permanently bonded to glass or other PDMS by treating both surfaces in the plasma cleaner for 15 s.

The pressure-actuated valves (microvalves) used with the microfluidic device in this example are illustrated in FIG. 16. The microvalves used in this example are a modification of a valve originally published by Unger et al. [M. A. Unger, H.-P. Chou, T. Thorsen, A. Scherer, and S. R. Quake, “Monolithic Microfabricated Valves and Pumps by Multilayer Soft Lithography,” Science, vol. 288, pp. 113-116, 2000] and modified by Studer et al. [V. Studer, et al., “Scaling properties of a low-actuation pressure microfluidic valve,” Journal of Applied Physics, vol. 95, pp. 393-398, 2004]. The pneumatic layer is cast on SU-8 negative photoresist (Microchem, Newton, Mass.), and the fluid layer is cast on AZ 100XT (Clariant, Somerville, N.J.), a positive photoresist that reflows into a rounded channel at high temperature. The membrane layer is made by spinning PDMS onto a glass wafer. The membrane layer also serves as the substrate for deposition of the ferromagnetic elements used to capture the yeast cells.

Cell Capture. Single cell capture is selective for the mother cell and allow the daughter cell to flow free after budding. The cell capture method also is size-independent because the daughter cell is the same size as the mother cell at the beginning and the end of the mother's life. A successful cell capture method is the magnetic capture of a biotinylated yeast cell attached to streptavidin-coated paramagnetic beads. If the mother cell is incubated with the vitamin biotin at room temperature, the biotin molecule will attach to proteins on the cell wall, a process called biotinylation. When a daughter cell forms, it creates a new cell wall [C. E. Ballou, “Yeast cell wall and cell surface,” in The molecular biology of the yeast Saccharomyces. Metabolism and gene expression., J. R. Broach, Ed. Cold Spring Harbor, N.Y.: Cold Spring Harbor Laboratory Press, 1982, pp. 335-360], and the daughter cell typically do not have this biotin coat [T. Smeal, J. Claus, B. Kennedy, F. Cole, and L. Guarente, “Loss of transcriptional silencing causes sterility in old mother cells of S-cerevisiae,” Cell, vol. 84, pp. 633-642, 1996]. Biotin forms a tight bond with the protein streptavidin. Streptavidin-coated paramagnetic beads are easily attached to biotinylated yeast by incubating them together for 15 min at room temperature. The yeast cell can then be captured by generating a magnetic force on the attached beads that is stronger than the drag force on the yeast cell, approximated as a spherical particle.

The Stokes drag force on the yeast cell is:
F_{d}=3πν_{c}D_{c}η (5)
where ν_{c} is the velocity of the yeast cell, D_{c} is the diameter of the yeast cell, and η is the viscosity of the surrounding medium. The direction of the force is opposite the direction of the velocity.

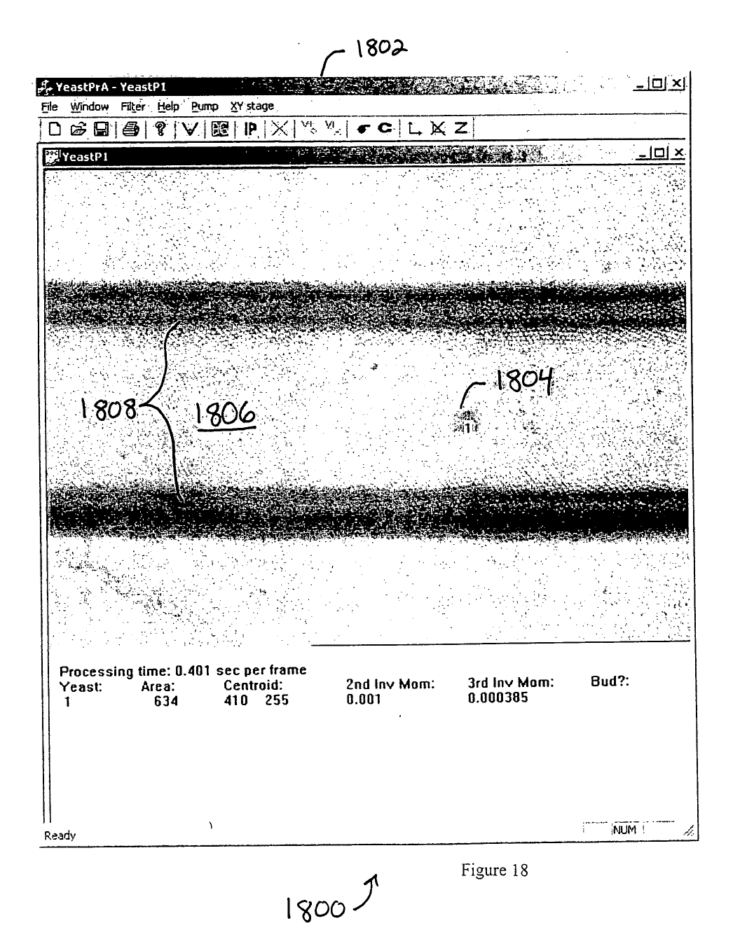

FIG. 18 is a display of a user interface system 1800 of an embodiment of the system of the present invention that provides an image 1802 of a microfluidic channel (channel walls shown as 1808, lumen shown as 1806) and a yeast cell 1804 flowing within the lumen 1806 of the microfluidic channel.

FIG. 19 illustrates the operation of a pressure-actuated valve 1900. The valve 1908 is normally open (A). To shut the valve, positive pressure is applied to the pneumatic layer 1902 to push the membrane 1904 against the ceiling of the fluid channel 1908 and close the channel (B).

The drag force is proportional to the velocity of the yeast cell; thus, cell velocity needs to be as low as possible to reduce the amount of magnetic force required. A flow of 1 nL/s in a typical 100 μm×50 μm channel results in a velocity of 40 μm/s and a drag force of approximately 1.5×10⁻¹² N.

The force on a single magnetic bead [K. E. McCloskey, J. J. Chalmers, and M. Zborowski, “Magnetic cell separation: Characterization of magnetophoretic mobility,” Analytical Chemistry, vol. 75, pp. 6868-6874, 2003] is:

$${F}_{b} = \frac{1}{2 {μ}_{0}} Δ χ {V}_{b} ∇ {B}^{2} , \qquad ( 6 )$$

where μ₀ is the magnetic permeability of free space, Δ_{χ} is the difference in magnetic susceptibility between the bead and the surrounding medium, V_{b} is the volume of the bead, and B is the magnetic flux density. The magnetic force is proportional to the gradient of the square of the magnetic flux density; therefore, a suitable magnet will generate both a strong field and a strong gradient.

One-dimensional MATLAB models of (5)-(6) have been examined using a cylindrical permanent magnet assuming conservative estimates for all parameters [J. Koschwanez, “Automation of Yeast Pedigree Analysis,” University of Washington, Seattle, MSEE Thesis, 2004]. Modeling shows that as the radius of a permanent magnet is reduced, there are two effects: the magnetic force increases non-linearly, and the peak force occurs closer to the tip of the magnet. The magnetic force required to capture the yeast cell is typically on the order of one piconewton (10⁻¹² N). A yeast cell coated with five 0.82 μm beads can use a less than 50 μm diameter permanent magnet less than 10 μm away from the channel to generate a piconewton of magnetic force. The model is approximate and makes a number of assumptions, but it provides an estimate of the scale of the magnet required to capture a bead.

The magnetic force is generated in the microfluidic device by externally magnetizing ferromagnetic elements that are microfabricated into the device. A series of successively smaller ferromagnetic elements have been fabricated using various methods [J. Koschwanez, “Automation of Yeast Pedigree Analysis,” University of Washington, Seattle, MSEE Thesis, 2004]. The following is a method of fabricating high-resolution cobalt-nickel-boron ferromagnetic elements on PDMS.

First, a clean glass slide is silanized in a desiccating chamber for 30 min. A 10-20 μm thick layer of PDMS is then spun onto the glass slide and cured for 2 hours in a 55° C. oven. The cured PDMS is treated in a plasma chamber for 20 s. The plasma treated PDMS is covered in 0.5 mM SnCl₂ for 5 min, rinsed with deionized water, covered in 0.5 mM SnCl₂ for 5 min, and again rinsed with deionized water. These initial steps serve to deposit palladium on the PDMS to act as the catalyst for electroless deposition.

The electroless deposition bath contained 0.05 M nickel sulfate, 0.07 M cobalt sulfate, 0.10 M trisodium citrate, and 0.024 M dimethylamineborane (DMAB) [T. S. N. S. Narayanan, A. Stephan, and S. Guruskanthan, “Electroless Ni—Co—B ternary alloy deposits: preparation and characteristics,” Surface & Coatings Technology, vol. 179, pp. 56-62, 2004]. 0.67 mM thiodiglycolic acid was added to stabilize the solution on the PDMS. The bath was heated to 48° C. and the pH was increased to 7.00 by addition of NaOH. The PDMS was covered in the bath for 8 min, then rinsed in DI water. At this point, the PDMS was covered in a thin layer of Co—Ni—B alloy.

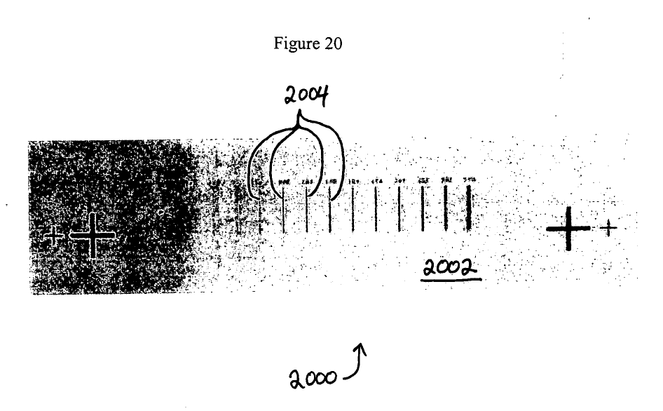

SIPR 7120-05 (ShinEtsu MicroSi, Phoenix, Ariz.), a positive photoresist, was patterned on the metal layer using standard photolithographic techniques. The metal not covered with photoresist was then stripped off using FeCl₃/HCl etchant (Transene, Danvers, Mass.). Finally, the photoresist was removed using N-methyl-2-pyrrolidone (NMP). FIG. 20 is a photomicrograph of Co—Ni—B ferromagnetic structures patterned on PDMS. The leftmost line is 20 μm wide. FIG. 20 shows the resulting layer of PDMS patterned with the metal alloy. The PDMS was aligned and bonded to the channel layer to create a microfluidic chip having a magnetic capture site.

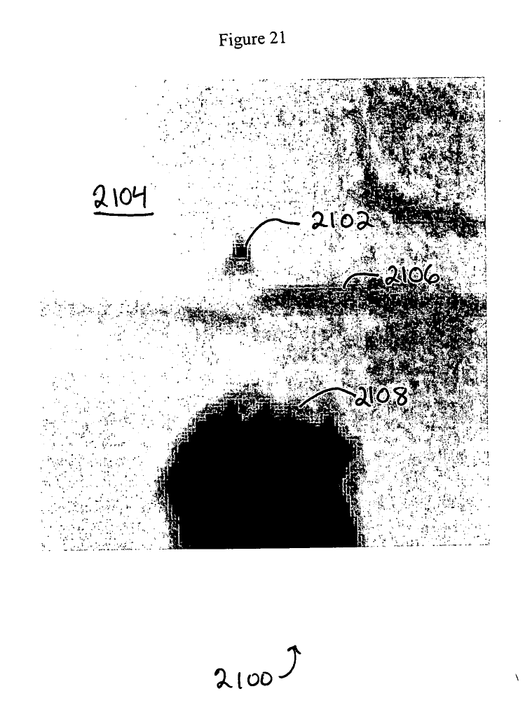

Results. Ferromagnetic structures with resolution better that 20 μm were fabricated on PDMS. A single yeast cell was held and released repeatedly with multiple magnetic elements. FIG. 21 shows an image 2100 of the successful capture of a single captured yeast cell 2102. The magnetic element 2108 is at the bottom of the image and the lower wall of the microfluidic channel 2106 is the horizontal line across the image. The imaging system and image processing software has been successfully tested and integrated into the automated system. The valves were successfully tested and are integrated into the microfluidic device. The output and input stations have been built and tested. The current software version controls all completed subsystems.

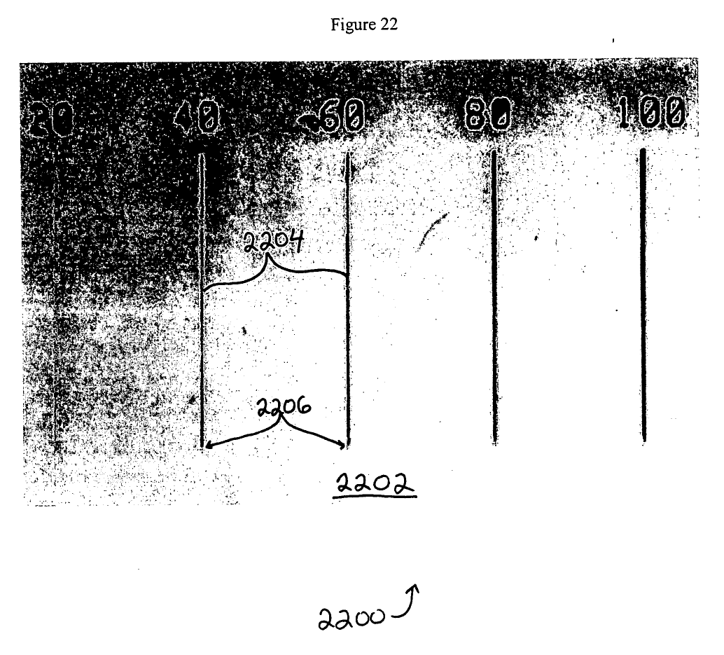

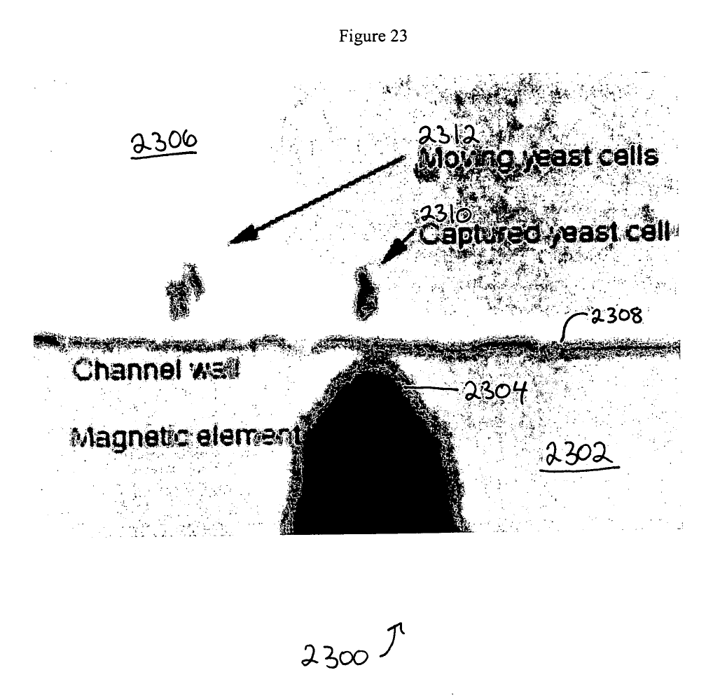

Example of Electrolessly Deposited Ferromagnetic Structures on PDMS. Electroless deposition methods are diagrammed in FIG. 6. The first method uses a photoresist as a deposition mask. After the metal is plated on the patterned photoresist, the photoresist is developed away to lift off the metal. In the second method, the entire surface of the PDMS is plated, and then the patterned photoresist serves as an etch mask. FIG. 22 shows ferromagnetic structures 2204 having ends 2206 for generating magnetic field gradients in a lumen of a microchannel (not shown). The ferromagnetic structures were made with the second method. The metal is a Ni—Co—B ferromagnetic alloy; the bath recipe and plating conditions are given in the paper. Both plating methods have been tested with Sylgard 184 PDMS (Dow Corning, Midland, Mich.) and GE RTV615 PDMS (General Electric, Waterford, N.Y.) in 5:1, 10:1, and 20:1 base to curing agent ratios. To construct a device for yeast cell capture, PDMS is cast on a mold of a 100 μm wide channel of AZ100XT photoresist, and then bonded to the PDMS layer containing the ferromagnetic structures by either plasma treating both surfaces or using complementary base to curing agent ratios. The layer containing the magnetic elements is then lifted off the glass wafer and bonded to a microscope slide or another layer of PDMS (in a multilayer device). The yeast is biotinylated, attached to streptavidin-coated 0.82 μm paramagnetic beads, and then pumped through the channel. The magnetic elements are magnetized using a Nd—Fe—B magnet placed on axis with the element. FIG. 23 is a still 2300 from a yeast cell capture video using the system described herein. Two yeast cells 2312, each budding, approach from the left in the flow channel. A yeast cell 2310, also budding, is held in place within the lumen 2306 of the microfluidic channel adjacent to the microfluidic channel wall 2308 opposite to the tip 2304 of the ferromagnetic structure (“magnetic element”). The ferromagnetic structures are characterized using atomic force microscopy (AFM) and optical profiling.

### Example

### Single Cell Capture with Ferromagnetic Elements Grown on PDMS

In this example, the magnetic capture and release of a single yeast cell using a system of the present invention is reported. This system was manufactured using a method of patterning and electrolessly depositing ferromagnetic elements directly onto polydimethylsiloxane (PDMS). The ease and speed of fabrication allowed the rapid building and testing of disposable devices. The deposition directly on PDMS allowed flexibility in placing the elements within a complex, multilayer microfluidic device and facilitates integration with on chip pumps, heaters, and valves.

Previously reported methods of fabricating magnetic traps for particle capture involve electroplating, evaporation, sputtering, or vapor deposition on a silicon or glass substrate (review in N. Pamme, Lab on a Chip, 6, pp. 24-38 (2006)). Metal placement on PDMS (not used for particle capture) has previously been reported using electron beam evaporation N. Bowden et al., Nature, 393, p. 146-149 (1998) or transfer from a silicon substrate K. S. Lim et al., Lab on a Chip, 6, pp. 578-580 (2006).

In this example, the ferromagnetic elements were electrolessly deposited directly onto PDMS at 50° C. This fabrication method has many advantages: (i) The fabrication is quick and inexpensive and can be performed outside a cleanroom (after the reusable stamp molds are fabricated). (ii) The element fabrication can be easily integrated into a series of fabrication steps. (iii) The ferromagnetic elements can be placed on any layer in a multilayer PDMS device, rather than on the glass or silicon substrate at the top or bottom of the device, and therefore (iv) the distance between the element and the captured cell is limited only by alignment precision and a protective biocompatible layer. Reducing this distance increases the magnetic force at the capture site.

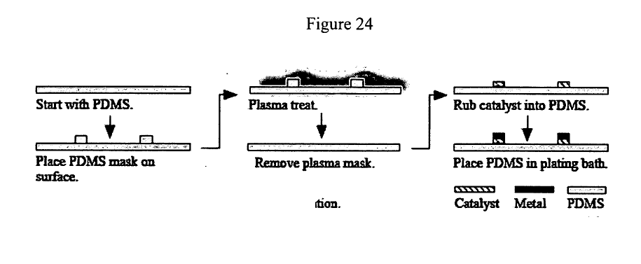

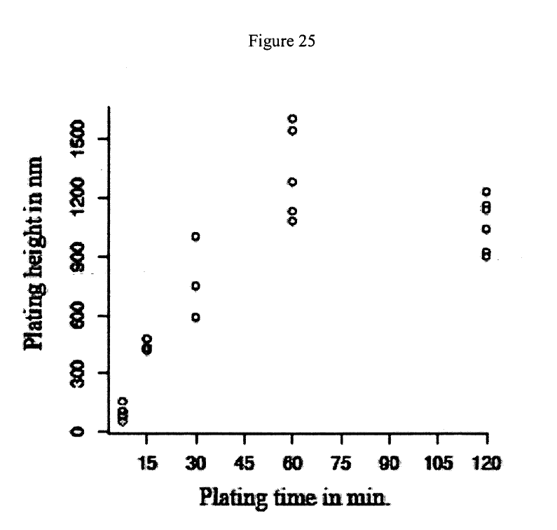

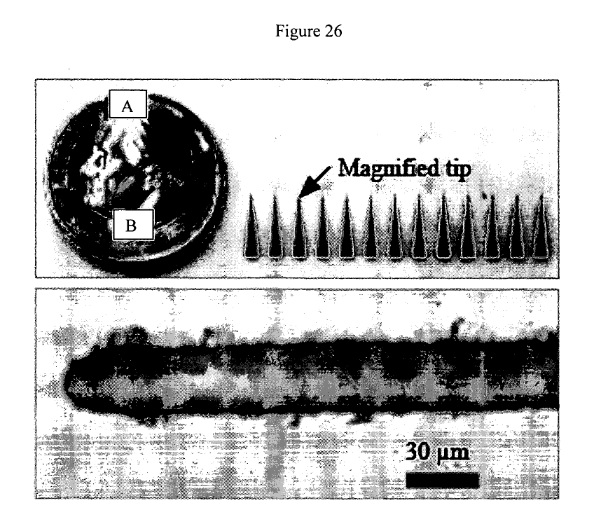

FIG. 24 shows the fabrication schematic. First, the PDMS was plasma treated; those areas that will be plated were shielded from plasma treatment by a PDMS stamp. Next, a palladium chloride catalyst was rubbed into the PDMS using vinyl foam; the catalyst adsorbs to those areas shielded from plasma treatment. The catalyst is then exposed to the nickel-cobalt plating bath for the desired time (FIG. 25). FIG. 26 shows elements fabricated using a triangular stamp with an extended tip.

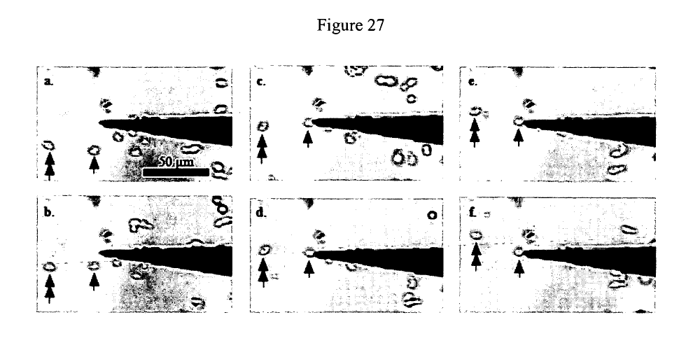

To construct the capture device, a protective layer of PDMS was spun on the magnets and a PDMS microfluidic channel is bonded above the magnets. Biotinylated yeast cells were attached to streptavidin-coated 50 nm paramagnetic beads and then pumped into the channel. 50 nm beads eliminated cell aggregation, unlike larger beads used in other cell trapping demonstrations. When the ferromagnetic elements were externally magnetized using a small permanent magnet, a cell was captured (FIGS. 27a-f). When the magnet was removed, the cell was released.

After the magnetic force was characterized, the ferromagnetic elements were integrated into a single cell analysis system as described herein.

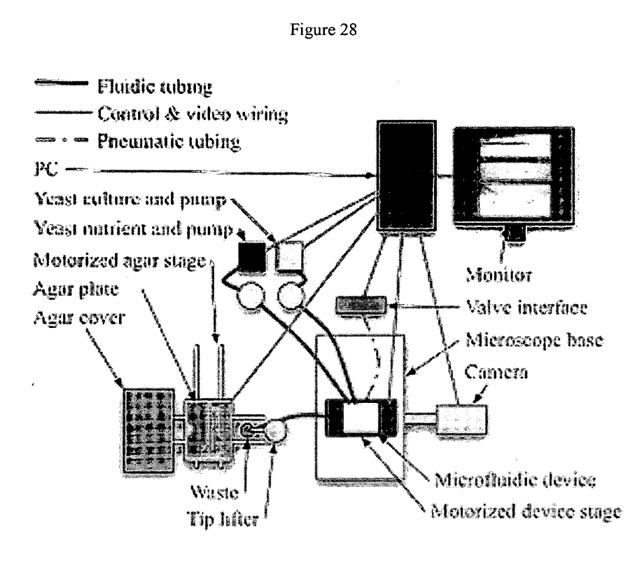

Automated Analysis System. FIG. 28 shows an example of an overall system architecture of a yeast automated lifetime analysis system which incorporates a microfluidic device that captures multiple mother cells and distributes their daughter cells to an agar plate. FIG. 28 is depicted without a fiber optic bundle, which is included in FIG. 15. The software, microfluidic device, and the controllable single cell trap are described further herein. The entire system is bolted to a vibration isolation table, and when complete will be enclosed in an acrylic box and heated to 30° C. for yeast growth. The microfluidic device sits on a motorized stage on an inverted microscope base, which holds the objective, filter cubes, and the prism to send light to the CCD camera. The device can be readily illuminated via transmitted light or by a halogen lamp or an LED, and it can be illuminated via reflected light by a mercury vapor arc lamp through a shutter and an epifluorescent attachment to the microscope base.

System operation. An automated system uses manual preparation of a yeast culture. A yeast colony from a YEPD (a yeast nutrient) agar plate is grown for approximately 12 hours in liquid YEPD at 30° C. When the yeast concentration reached about 10⁷ cells/mL, the yeast were biotinylated and attached to 50 nm streptavidin coated paramagnetic beads (Miltenyi Biotech, Germany). Streptavidin is a protein that binds very tightly to biotin. The loose paramagnetic beads were then separated using sucrose gradients, and the culture was diluted to about 10⁵-10⁶ cells/mL. Cell concentration was determined using a glass slide hemacytometer. As a result of cell division, the cell culture contained 50% new daughter cells, 20% mothers of age one, 12.5% mothers of age two, and so on.

FIG. 4 shows a schematic of multiple capture sites in one microfluidic channel. The microfluidic channels were placed in parallel on the device, and each channel has one inlet and one outlet valve, allowing selective release of daughters in separate channels. The yeast culture bottle was attached to the pump inlet at the input of the automated system. All remaining actions were automated.

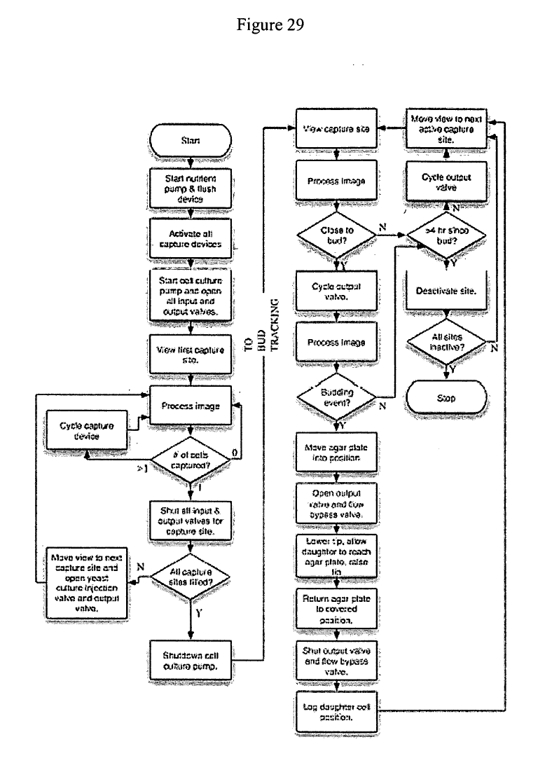

FIG. 29 shows the system flow diagram. The nutrient pump was started to flush all air out of the device and prime all channels with liquid. Each output valve is opened and each cell culture injection valve is opened. Each capture device is actuated and the cell culture pump is started. The agar plate is under its cover and the output tubing drips to waste. Cell culture is now flowing by every capture device to waste. The image of the first capture site is viewed and processed to determine if capture has taken place. If greater than one cell is captured at a single capture device, the capture device is released and re-actuated. If one cell is captured at each capture device, the output and input valves for the site are shut. The view is shifted to the new capture site. When all capture sites have captured cells, the cell culture pump is shut down and the bud tracking cycle is started.

The mother yeast cell will bud every 90 minutes while it is trapped. To start the bud tracking cycle, the first capture site is viewed. The image is processed to determine if the cell is close to budding. If a budding event will occur soon, the output valve is open and shut to attempt to free the daughter cell from the mother cell. A budding even has occurred when the software has determined that the daughter cell has separated from the mother cell. If the budding event has occurred, the agar plate is aligned to the correct loacation using an xy-stage that is moved by two stepper motors (In between buds, the agar plate is covered to maintain hydration of the media.) The output valve is then opened and the daughter cell flows with the nutrient, through the output channel, through the output PEEK tubing held by the tip lifter, and to the designated location on the agar plate. The tip lifter, which moves in the z-direction only, drops the tip of the PEEK tubing to within 1 mm of the agar plate. Surface tension pulls the drop containing the daughter yeast cell onto the agar plate. Then the output valve is shut, the tip is raised, and the agar plate is returned to its standby, covered position. The daughter cell location is logged and the view is shifted to the next active capture site. When it has been more than 4 hours since the most recent budding event at a capture site, the site is labeled inactive. When all sites are labeled inactive, the operation is complete. The agar plate with the daughter colonies can be removed for further analysis.

The time required to image a site and move the device to the next position is approximately 1 second (“s”). The time required to send a daughter cell to the agar plate is approximately 10 s. In a multiple cell device, there is the possibility that unwatched cells could bud while monitoring another capture site. Cycling valves only while monitoring the capture site prevents sending an unwatched daughter cell to the agar plate.

Software. The software automates the yeast pedigree analysis system by processing the images, determining the budding status of the yeast cell, and controlling the input pumps, valves, motorized device stage, motorized agar plate stage, motorized filter cube changer, camera, shutter, and tip lifter. The software is suitably written in C++ in the Microsoft (Redmond, Wash.) Visual C++.NET development environment. The software can run on a personal computer with a Microsoft Windows XP operating system.

Imaging system. Novel imaging processing and analysis software has been written to filter out noise, recognize, a yeast cell and classify it as budding or not budding a yeast cell as described further herein. In addition, the software can be used when a fiber-optic bundle is part of the optical imaging train as further described herein.

Microfluidic device. Microfluidics replaces manual cell manipulation by providing the means of delivering a yeast cell to the capture site and the daughter cell to the agar plate. Poly(dimethylsiloxane) (PDMS, Dow Corning, Midland, Mich. and RTV 615, GE, Fairfield, Conn.) was chosen as the material for the microfluidic device because it is optically transparent, it can easily be bonded to itself, and its elasticity allows its use as a valve membrane material. In addition, the surface can be changed from hydrophobic to hydrophilic by treating it for 15 s or longer in a plasma cleaner (Harrick, Ossining, N.Y.). G. S. Fiorini and D. T. Chiu, “Disposable microfluidic devices: fabrication, function, and application,” BioTechniques, vol. 38, pp. 429-446, 2005, describes microfluidic fabrication techniques and applications which can be adapted for use in the present invention.

PDMS microfluidic devices are fabricated using a rapid prototyping method developed by Whitesides and coworkers [D. C. Duffy, J. C. McDonald, O. J. A. Schueller, and G. M. Whitesides, “Rapid Prototyping of Microfluidic Systems in Poly(dimethylsiloxane),” Anal. Chem., vol. 70, pp. 4974-4984, 1998]. A mask can be drawn using a computer aided drafting (CAD) program and printed on a high-resolution printer. The photoresist is spun on a silicon wafer and exposed to UV light through the mask. The unexposed (negative) or exposed (positive) photoresist is then washed away using the photoresist developer. PDMS is poured and cured on the remaining structure to create microfluidic channels. The layer of PDMS can be permanently bonded to glass or other PDMS by treating both surfaces in the plasma cleaner for 15 s or by alternating base to catalyst ratios in the PDMS [M. A. Unger, H.-P. Chou, T. Thorsen, A. Scherer, and S. R. Quake, “Monolithic Microfabricated Valves and Pumps by Multilayer Soft Lithography,” Science, vol. 288, pp. 113-116, 2000].

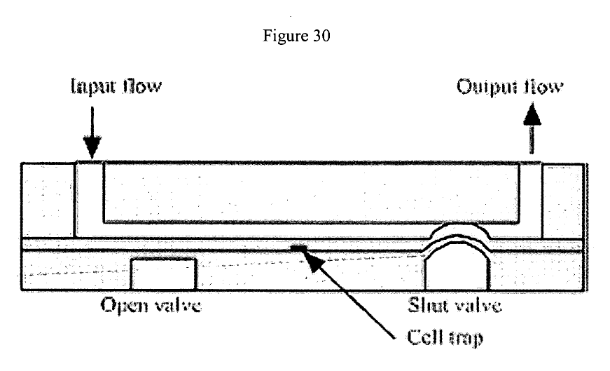

FIG. 30 shows a simple single cell capture device with one inlet and one outlet. The pressure-actuated valves used with the device are a modification of a valve originally published by Unger et al. [M. A. Unger, H.-P. Chou, T. Thorsen, A. Scherer, and S. R. Quake, “Monolithic Microfabricated Valves and Pumps by Multilayer Soft Lithography,” Science, vol. 288, pp. 113-116, 2000] and modified by Studer et al. [V. Studer, G. Hang, A. Pandolfi, M. Ortiz, W. F. Anderson, and S. R. Quake, “Scaling properties of a low-actuation pressure microfluidic valve,” Journal of Applied Physics, vol. 95, pp. 393-398, 2004]. The pneumatic layer is spun on a wafer with SU-8 negative photoresist (Microchem, Newton, Mass.), and the fluid layer is cast on AZ 100XT (Clariant, Somerville, N.J.), a positive photoresist that reflows into a rounded channel at high temperature. The pneumatic layer also serves as the substrate for deposition of the ferromagnetic elements used to capture the yeast cells. A partially cured layer bonds the two layers together.

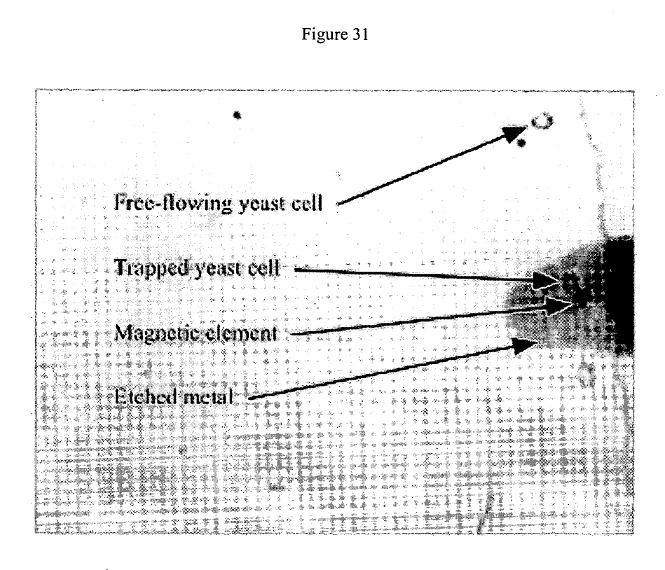

Cell capture. Single cell capture, shown in FIG. 31, can select for a mother cell and allow a daughter cell to flow free after budding. This cell capture method can be size independent as the daughter cell is the same size as the mother cell at the beginning and the end of the mother's life. Here, the paramagnetic bead-labeled yeast cell is captured at the tip of the externally magnetized element. The magnetic cell trap can use paramagnetic beads to be attached to the cell. First, the mother cell is incubated with the vitamin biotin at room temperature. The biotin molecule attaches to proteins on the cell wall, a process called biotinylation. When a daughter cell forms, it creates a new cell wall [C. E. Ballou, “Yeast cell wall and cell surface,” in The molecular biology of the yeast Saccharomyces. Metabolism and gene expression., J. N. Strathern, E. W. Jones, and J. R. Broach, Eds. Cold Spring Harbor, N.Y.: Cold Spring Harbor Laboratory Press, 1982, pp. 335-360]. A daughter cell typically does not have this biotin coat [T. Smeal, J. Claus, B. Kennedy, F. Cole, and L. Guarente, “Loss of transcriptional silencing causes sterility in old mother cells of Scerevisiae,” Cell, vol. 84, pp. 633-642, 1996]. Biotin forms a tight bond with the protein streptavidin. Streptavidin-coated paramagnetic beads are easily attached to biotinylated yeast by incubating them together for 15 min at room temperature. Various sizes of paramagnetic beads can be used. The yeast cell can then be captured by generating a magnetic force on the attached beads that is stronger than the drag force on the yeast cell.

When the paramagnetic bead encounters a magnetic field, a magnetic moment is induced in the bead proportional to the magnetic field and the volume of the bead. If the field is nonuniform, a force can be generated proportional to the gradient of the magnetic field and the magnetic moment. The resulting force on a single magnetic bead [K. E. McCloskey, J. J. Chalmers, and M. Zborowski, “Magnetic cell separation: Characterization of magnetophoretic mobility,” Analytical Chemistry, vol. 75, pp. 6868-6874, 2003] can be proportional to the gradient of the square of the magnetic flux density; therefore, the magnet usually generates both a strong field and a strong gradient. Modeling of externally magnetized ferromagnetic elements has shown that a strong field gradient in the microfluidic channel can be generated by narrow triangular ferromagnetic tips placed next to the channel.

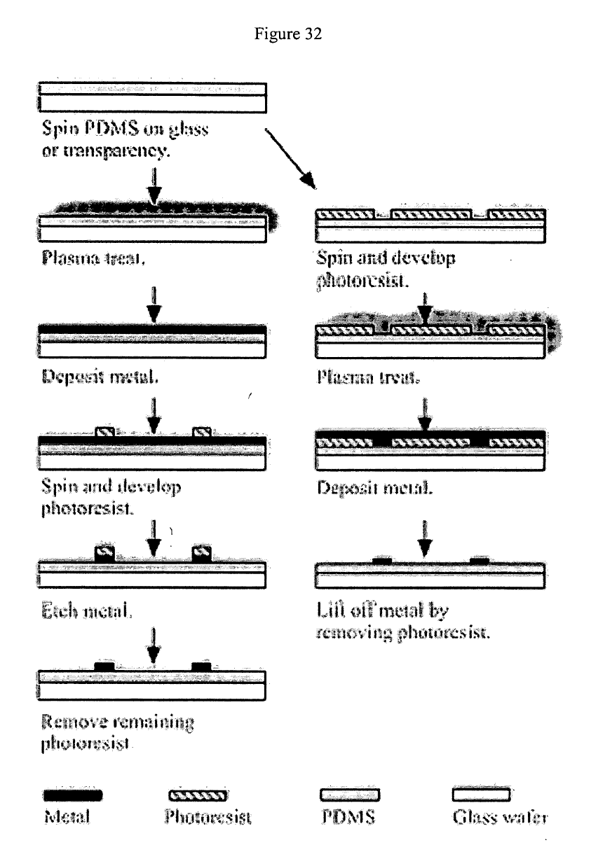

A magnetic force is generated in the microfluidic device by externally magnetizing ferromagnetic elements that are microfabricated into the device. A series of successively smaller ferromagnetic elements can be fabricated as described herein. Two electroless deposition methods developed in our lab are diagrammed in FIG. 32. The first method uses a photoresist as a deposition mask. After the metal is plated on the patterned photoresist, the photoresist is developed away to lift off the metal. In the second method, the entire surface of the PDMS is plated, and then the patterned photoresist serves as an etch mask. The metal is a Ni—Co—B ferromagnetic alloy based on the bath developed by Narayanan et al. [T. S. N. S. Narayanan, A. Stephan, and S. Guruskanthan, “Electroless Ni—Co—B ternary alloy deposits: preparation and characteristics,” Surface & Coatings Technology, vol. 179, pp. 56-62, 2004]. Both plating methods have been tested with Sylgard 184 PDMS (Dow Corning, Midland, Mich.) and GE RTV615 PDMS (General Electric, Waterford, N.Y.) in 5:1, 10:1, and 20:1 base to curing agent ratios.

To construct a device for yeast cell capture, PDMS is cast on a mold of a 100 μm wide channel of AZ100XT photoresist [D. C. Duffy, J. C. McDonald, O. J. A. Schueller, and G. M. Whitesides, “Rapid Prototyping of Microfluidic Systems in Poly(dimethylsiloxane),” Anal. Chem., vol. 70, pp. 4974-4984, 1998], and then bonded to the PDMS layer containing the magnet elements by either plasma treating both surfaces or using complementary base to curing agent ratios [M. A. Unger, H.-P. Chou, T. Thorsen, A. Scherer, and S. R. Quake, “Monolithic Microfabricated Valves and Pumps by Multilayer Soft Lithography,” Science, vol. 288, pp. 113-116, 2000]. The layer containing the magnetic elements is then lifted off the glass wafer and bonded to a microscope slide or another layer of PDMS (in a multilayer device). The yeast is biotinylated, attached to streptavidin-coated 50 nm paramagnetic beads, and then pumped through the channel. The magnetic elements are magnetized using a Nd—Fe—B magnet placed on axis with the element.

Results. The imaging system and image processing software have been successfully tested and integrated into the automated system [J. Koschwanez, M. Holl, B. Marquardt, J. Dragavon, L. Burgess, and D. Meldrum, “Identification of budding yeast using a fiber-optic imaging bundle,” Review of Scientific Instruments, vol. 75, pp. 1363-1365, 2004; J. Koschwanez and D. Meldrum, “Picture Perfect,” in oe magazine, vol. 4, 2004, pp. 28-30]. The valves have been successfully tested and are integrated into the device. The current software version controls all completed subsystems.

Magnetic elements with resolution of 20 μm have been fabricated on PDMS. A single yeast cell attached to 50 nm paramagnetic beads has been held and released with multiple magnetic elements. FIG. 31 shows a successful capture.

One or more magnetic elements are used to hold a single yeast cell in place for its lifetime. After this, the system components are integrated into an automated system. After successful single cell testing, the system is expanded to perform yeast pedigree analysis on multiple yeast cells on the same device. At the same time, a fluorescence analysis system is used to monitor parameters of the mother cell over its lifetime.

1. A method, comprising:
providing a biologically-compatible substrate comprising a surface;
depositing an electroless deposition catalyst on the surface of the biologically-compatible substrate; and
electrolessly depositing a ferromagnetic composition on the surface of the biologically-compatible substrate.

2. The method of claim 1, further comprising the step of lithographically patterning the ferromagnetic composition on the surface of the biologically-compatible substrate.

3. The method of claim 2, wherein lithographically patterning the ferromagnetic composition includes at least one of photolithography, imprint lithography, or any combination thereof.

4. A method, comprising:
exposing the surface to a plasma;
optionally applying a sensitizer to the surface;
depositing an electroless deposition catalyst on the surface; and
electrolessly depositing a ferromagnetic composition on the surface of the substrate.

5. The method of claim 4, wherein the substrate is PDMS.

6. The method of claim 5, further comprising the step of patterning the ferromagnetic composition with at least one of photolithography or imprint lithography.

7. A magnetic capture site comprising:
a microchannel disposed on a substrate; and
one or more ferromagnetic structures disposed on or within the substrate, the ferromagnetic structures comprising a tapered end and a body, wherein the tapered end of at least one of the ferromagnetic structures is proximately located to one or more lumens of one or more of the microchannels, the ferromagnetic structures capable of being magnetized using a magnetic field source proximately located external to the body of the ferromagnetic structure.

8. The magnetic capture site of claim 7, wherein the tapered end comprises a triangle, a point, a corner, a narrow rectangle comprising one or more point-like tips, or any combination thereof.

9. The microfluidic device of claim 7, wherein the tapered end is located external to the lumens of the microchannels.

10. The microfluidic device of claim 7, wherein the magnetic field source is located external to the microfluidic device.

11. A microfluidic device comprising a magnetic capture site capable of magnetically capturing a magnetically-labeled microorganism, cell, particle, molecule, or any combination thereof, the magnetic capture site comprising:
one or more microchannels disposed on the microfluidic device; and
one or more ferromagnetic structures disposed on or within the microfluidic device, the ferromagnetic structures comprising a tapered end and a body, wherein the tapered end of at least one of the ferromagnetic structures is proximately located to one or more lumens of one or more of the microchannels, the ferromagnetic structures capable of being magnetized using a magnetic field source proximately located external to the body of the ferromagnetic structure.

12. The microfluidic device of claim 11, wherein the tapered end comprises a triangle, a point, a corner, a narrow rectangle comprising one or more point-like tips, or any combination thereof.

13. The microfluidic device of claim 11, wherein the tapered end is located external to the lumens of the microchannels.

14. The microfluidic device of claim 11, wherein the magnetic field source is located external to the microfluidic device.

15. A method, comprising:
providing a microfluidic device comprising one or more microchannels and one or more ferromagnetic structures disposed within the microfluidic device, at least one of the ferromagnetic structures located in proximity to one or more lumens of one or more of the microchannels;
fluidically transporting a magnetically-labeled microorganism, a magnetically-labeled cell, a magnetically-labeled particle, or a magnetically-labeled molecule through one of the lumens and towards one of the ferromagnetic structures;
controllably magnetizing one of the ferromagnetic structures to create a magnetic field passing through a portion of one or more lumens; and
controllably holding the magnetically-labeled microorganism, the magnetically-labeled cell, the magnetically-labeled particle, or the magnetically-labeled molecule using the magnetic field within one of the lumens.

16. The method of claim 15, wherein the magnetically-labeled microorganism includes a magnetically-labeled yeast, a magnetically-labeled bacteria or a magnetically-labeled virus.

17. The method of claim 15, wherein the magnetically-labeled cell includes a eukaryotic cell or a prokaryotic cell.

18. The method of claim 15, wherein the magnetically-labeled particle includes a magnetically-labeled vesicle, a magnetically-labeled liposome, or a magnetically-labeled macromolecular complex.

19. The method of claim 15, wherein the magnetically-labeled molecule includes a magnetically-labeled nucleic acid, a magnetically-labeled amino acid, a magnetically-labeled carbohydrate, or any combination thereof.

20. The method of claim 15, wherein the lumen is in fluid communication with an inlet and an outlet of the microchannel,
a magnetically-labeled microorganism, a magnetically-labeled cell, a magnetically-labeled particle, or a magnetically-labeled molecule and a fluid medium flow into the lumen through the inlet,
the magnetically-labeled microorganism, the magnetically-labeled cell, the magnetically-labeled particle, or the magnetically-labeled molecule is controllably held using the magnetic field in the lumen, and
the fluid medium flows out of the outlet.

21. The method of claim 20, wherein the fluid medium comprises compounds or nutrients capable of being absorbed or metabolized by the magnetically-labeled organism or magnetically-labeled cell.

22. The method of claim 20, wherein the fluid medium comprises compounds capable of conjugating, binding, complexing, hybridizing, or any combination thereof, with the magnetically-labeled particle or molecule.

23. The method of claim 20 wherein the fluid medium flows past the magnetically-labeled microorganism, cell, particle, or molecule through the lumen while the magnetically-labeled microorganism, cell, particle, or molecule is controllably held.

24. The method of claim 15, wherein the magnetically-labeled microorganism or cell produces one or more daughter cells as it is controllably held.

25. The method of claim 24, wherein the one or more daughter cells are fluidically transported to one or more collection receptacles.

26. The method of claim 24 further comprising the step of magnetically labeling at least one of the daughter cells.

27. The method of claim 26, wherein the daughter cells are magnetically labeled in a microfluidic mixing chamber.

28. The method of claim 24, further comprising repeating the following steps sequentially one or more times:
optionally magnetically-labeling a daughter cell;
fluidically transporting the magnetically-labeled daughter cell through the lumen and towards a second magnetic structure;
controllably magnetizing the second magnetic structure to create a second magnetic field; and
controllably holding the magnetically-labeled daughter cell with the second magnetic field.

29. The method of claim 28 wherein the second magnetic structure is located down stream from the ferromagnetic structure where the daughter cell was produced.

30. The method of claim 15 further comprising the steps of:
imaging the magnetically labeled cell as it is being controllably held by the magnetic field within the lumen;
processing the image with an image processor, the image processor determining the production of a daughter cell by the magnetically labeled cell; and
fluidically transporting the daughter cell to a second magnetic structure or a second microfluidic channel.

31. The method of claim 15, wherein one or more of the ferromagnetic structures are each capable of creating a magnetic field passing through a portion of the lumens of two separate microchannels.

32. The method of claim 15, further comprising the step of controllably releasing the magnetically-labeled microorganism, cell, particle, or molecule from the magnetic field.

33. The method of claim 32, wherein the released cell is fluidically transported to a second microchannel, an agar plate, a test tube, a vial, a multiwell plate, a microreactor, a valve, an imaging area, or any combination thereof.

34. A method, comprising:
magnetically capturing one or more magnetically-labeled cells in a microfluidic device comprising one or more magnetic capture sites optically coupled to an imaging system, each of the magnetic capture sites fluidically coupled to one or more collection receptacles;
detecting the generation of a daughter cell by at least one of the magnetically-labeled cells using the imaging system; and
fluidically transporting the daughter cell from the microfluidic device to the one or more collection receptacles.

35. A system, comprising:
a microfluidic device comprising one or more magnetic capture sites optically coupled to an imaging system, wherein the magnetic capture sites are each capable of magnetically capturing a magnetically-labeled mother cell;
each of the magnetic capture sites being fluidically coupled to one or more collection receptacles;
wherein the imaging system is capable of detecting the generation of a daughter cell by the magnetically-labeled mother cell and actuating the microfluidic device to fluidically transport the daughter cell to the one or more collection receptacles.

36. The system of claim 35, wherein the microfluidic device comprises up to about 1,000 magnetic capture sites.

37. The system of claim 35, wherein the magnetic capture sites comprise:
one or more microchannels disposed on the microfluidic device; and
one or more ferromagnetic structures disposed within the microfluidic device, wherein at least one of the ferromagnetic structures are proximately located to one or more lumens of one or more of the microchannels.

38. The system of claim 35, wherein at least a portion of the magnetic capture sites are disposed on or within a biologically-compatible substrate.

39. The system of claim 38, wherein the magnetic capture sites are capable of being actuated by a magnetic field source located external to the magnetic capture site.

40. The system of claim 39, wherein the magnetic capture sites are capable of being externally actuated using a permanent magnet or an electromagnet, wherein the permanent magnet or electromagnet comprises a microfabricated structure disposed on or within the microfluidic device.

41. The system of claim 35, wherein the imaging system comprises at least one optical lens, at least one optical fiber imaging bundle, at least one optical fiber, at least one optical detector, at least one image processor, at least one optical filter, at least one mirror, at least one light source, or any combination thereof.

42. The system of claim 41, wherein the optical fiber imaging bundle is positioned to optically transmit an image of at least one of the magnetic capture sites.

43. The system of claim 35, wherein the imaging system comprises an optical train for transmitting at least one image of the at least one magnetic capture sites to an image processor.

44. The system of claim 43, wherein the image processor comprises a processor and an image processing algorithm.

45. The system of claim 35, wherein the imaging system comprises at least one light source positioned to illuminate the one or more magnetic capture sites, an optical train for transmitting one or more images of the magnetic capture site to an optical detector, the optical detector capable of communicating the image to an image processor.

46. The system of claim 45, wherein the magnetic capture site is situated between the light source and the optical train.

47. The system of claim 35, wherein the microfluidic device comprises a first substrate fluidically sealed to a second substrate, and the microfluidic device is optically transparent and capable of transmitting an image of the one or more magnetic capture sites through the first substrate, the second substrate, or both.

48. The system of claim 47, wherein the image is optically transmitted to the imaging system.

49. The system of claim 48, wherein the imaging system is capable of being spatially translated to individually receive an image of more than one of the magnetic capture sites.

50. The system of claim 35 further comprising one or more spatial translation devices for spatially positioning the microfluidic device, at least a portion of the imaging system for receiving an image of the one or more magnetic capture sites, or any combination thereof.

51. The system of claim 41, wherein the optical fiber imaging bundle comprises from about 100 to about 100,000 fiber cores having a diameter in the range of from about 1 μm to about 10 μm.

52. The system of claim 41, wherein the imaging system filters out noise introduced by the fiber optical bundle.

53. The system of claim 52, wherein the imaging system filters out the noise introduced by the fiber optical bundle by: capturing a gray-scale image of one or more magnetic capture sites;
smoothing the gray-scale image; converting the smoothed gray-scale image to a binary image using a locally generated threshold based on the mean and standard deviation of the subimage;
and segmenting the binary image into regions of pixels.

54. The system of claim 35 wherein the imaging system detects division progress of the mother cell and the generation of the daughter cell.

55. The system of claim 35, wherein the imaging system detects the division progress of the mother cell and the generation of the daughter cell by the following steps:
capturing a gray-scale image of one or more capture sites;
calculating the area of each region of dark pixels that is a potential cell;
removing the regions of dark pixels smaller than the area of a mother cell from the binary image;
and classifying each remaining region of dark pixels as being composed of a mother cell or of a mother cell and a daughter cell.

56. The method of claim 4, wherein the step of depositing an electroless deposition catalyst on said surface includes transferring catalyst from a second compliant substrate surface to said surface.
